(19) United States
(12) Patent Application Publication
(10) Pub. No.: US 2022/0372074 A1
(43) Pub. Date: Nov. 24, 2022

(54) Production and Purification Method for Polypeptide

(71) Applicant: South China University of Technology, Guangzhou (CN)

(72) Inventors: Zhanglin LIN, Guangzhou (CN); Yanyun JING, Guangzhou (CN); Xiaofeng YANG, Guangzhou (CN); Lei ZHAO, Guangzhou (CN); Peguy Paulie AMESSO NDENGUE, Guangzhou (CN)

(73) Assignee: South China University of Technology, Guangzhou (CN)

(21) Appl. No.: 17/772,104

(22) PCT Filed: Oct. 30, 2020
(86) PCT No.: PCT/CN2020/125054

§ 371 Date: Apr. 26, 2022

(30) Foreign Application Priority Data
Oct. 31, 2019 (CN) 201911053169.2

(51) Int. Cl. C07K 7/06; C07K 14/61; C12N 15/70; C12N 15/62; C07K 14/56

(52) U.S. Cl. CPC C07K 7/06; C07K 14/61; C12N 15/70; C12N 15/62; C07K 14/56; C07K 2319/00

(57) ABSTRACT

The present invention provides a fusion polypeptide comprising a target polypeptide moiety and a self-aggregating peptide moiety, and a method of producing and purifying a target polypeptide by expressing the fusion polypeptide.

### TECHNICAL FIELD

The present disclosure relates to the field of genetic engineering, and particularly to a fusion polypeptide comprising a target polypeptide moiety and a self-aggregating peptide moiety, and a method for producing and purifying the target polypeptide by expressing the fusion polypeptide.

### BACKGROUND

At present, the research and development of the application of polypeptides in medicine has widely involved antitumor drugs, cardiovascular and cerebrovascular drugs, vaccines and antiviral drugs, as well as diagnostic kits and other aspects (Leader et al., 2008). With the rapid growth of market demand, the production method of polypeptides limits its development. When the conventional chemical solid-phase synthesis method is used to produce medium-length polypeptides with more than 30 amino acids, the cost and difficulty of the synthesis will be greatly increased with the increase in the length of the peptides (Bray et al., 2003).

Another effective means is to use recombinant methods to produce polypeptides in host cells. In the methods for recombinant production of polypeptides, the purification step is very critical. It has been reported that the cost of isolation and purification of recombinant polypeptides accounts for about 60%-80/o of the total production cost (Chen Hao et al., 2002). The purification methods of recombinant polypeptides comprise conventional ion exchange chromatography, hydrophobic interaction chromatography, affinity chromatography, etc. Ion exchange chromatography and hydrophobic interaction chromatography are less versatile and efficient than affinity chromatography due to certain requirements for the initial conditions of the sample. Affinity purification can often achieve a high yield which is more than 90%, making it the most common method for the purification of recombinant proteins. Commonly used affinity purification techniques include fusion expression of target polypeptide with histidine tag (his-tag) or glutathione transferase tag (GST-tag), providing a universal purification method for the production of different target polypeptides. However, the expensive purification columns make the affinity purification high cost, which is not conducive to the application in the industrial field.

Human growth hormone (hGH) is a protein hormone secreted by the anterior pituitary gland. Its mature form is a non-glycosylated hydrophilic globulin with the signal peptide removed, consists of 191 amino acids and has two disulfide bonds and a relative molecular weight of about 22 kDa. hGH can arrive at various organs and tissues of the human body through the blood circulatory system, and its receptors are also throughout various cells in the human body, and thus, the growth hormone can act on almost all tissues and cells. hGH plays many important roles in the human body, such as maintaining positive nitrogen balance physiologically and initiating protein synthesis in muscle cells, increasing amino acid uptake in skeletal muscle, regulating longitudinal growth of bones, protecting cardiomyocytes and lymphocytes from apoptosis, etc. (Levarski et al., 2014; Zamani et al., 2015). Therefore, hGH has been widely used in the treatment of various diseases, and growth hormone has been approved by the US FDA for 11 indications. The approved indications in China mainly include 6 ones: growth hormone deficiency in children, growth hormone deficiency caused by burn symptoms and hypothalamic-pituitary disease, Tuner syndrome, adult human growth hormone deficiency, and chronic renal insufficiency. Currently, the global sale of growth hormone exceeds 3 billion US dollars. In China, the incidence of dwarfism in children is about 3%, and there are about 7 million patients, where the estimated market capacity exceeds 10 billion.

There are two main sources of growth hormone hGH for clinical application, direct extraction method and traditional genetic engineering method. The extraction from the pituitary glands is necessary for the direct extraction method, where the yield is low and the price is high, and thus, it cannot meet the needs of a large number of medical applications. It is forbidden due to greater safety risk. In traditional genetic engineering methods, prokaryotic expression systems are used for the production due to the aglycosylation of hGH, in which recombinant E. coli is mostly used. However, when expressed directly in E. coli cells, hGH is present in the form of inactive inclusion body, and the subsequent renaturation is required to obtain biologically active growth hormone hGH. At present, fusion tags are mainly used for solubilization (such as glutathione fragments, TNFα, etc.) (Levarski et al., 2014; Nguyen et al., 2014) or for periplasmic space expression (MBP tags) (Wang Kuqiang et al., 2018). These techniques and processes require more complicated purification steps and need to use a variety of column chromatography techniques, such as affinity chromatography, gel exclusion chromatography, etc., where the yield is low, and the cost is high, resulting in high prices of human growth hormone hGH products.

Human interferon-α2a belongs to type I interferon, which is a multifunctional and highly active inducible protein produced by leukocytes and lymphocytes. It consists of 165 amino acids and contains two pairs of intramolecular disulfide bonds. The relative molecular weight is about 19.2 kDa. Recombinant human interferon α2a has a broad-spectrum antiviral effect, and its antiviral mechanism is mainly the induction of the synthesis of variety of antiviral proteins such as 2-5 (A) synthase, protein kinase PKR, MX protein in target cells through the binding of interferon to the interferon receptor on the surface of target cells, thereby preventing the synthesis of viral proteins and inhibiting the replication and transcription of viral nucleic acids (Sen G C et al., 1992; Markus H. Heim et al., 1999). Interferon also has multiple immunomodulatory effects, which can improve the phagocytic activity of macrophages and enhance the specific cytotoxicity of lymphocytes against target cells, and promote and maintain the body immune surveillance, immune protection and immune homeostasis. Recombinant human interferon preparations are currently internationally recognized effective drugs for the treatment of hepatitis B and C. According to statistics from the National Health and Family Planning Commission, there are about 350 million hepatitis B virus carriers in the world, and about 100 million are in China (accounting for 29%, with more than 30 million patients), and China accounts for half of the about 700,000 viral hepatitis-related deaths in the world every year. In addition, recombinant human interferon is also approved in China for the treatment of chronic granulocyte, hairy cell leukemia, kidney cancer, melanoma and the like.

The early interferon is extracted from human leukocytes by purification technology, which is not only difficult due to source, complex process but also low yield, expensive, and has the possibility of potential blood-borne virus contamination. Until the mid-1970s, with the development of biomedicine and the emergence of genetic recombination technology, interferon is gradually produced through the fermentation production process of genetically engineered E. coli. However, inactive inclusion body is mainly obtained, and then, biologically active interferon is obtained through the process of denaturation and renaturation, and the interferon obtained by this method has a methionine residue at the N-terminus.

In recent years, studies have indicated that the fusion expression of target proteins, intein and self-assembling short peptide can induce the fusion protein to form an active protein aggregate, and the aggregate releases the target proteins into the supernatant through the self-cleavage of intein (Wu Wei et al., 2011; Xing Lei et al., 2011; Zhou Bihong et al., 2012). Although the separation and purification method of the protein is low-cost, simple to operate, and has good application prospects in industrial production, it has been reported in the prior art that this method is only suitable for the production of proteins without a disulfide bond. However, many important drugs such as human growth hormone, interferon α2a or the like have two disulfide bonds (structure information of human growth hormone can be found in the database UniProt with the access number P01241, https://www.uniprot.org/uniprot/P01241; structural information of interferon α2a can be found in the database UniProt with accession number P01563, https://www.uniprot.org/uniprot/PO1563). In order to solve the problem caused by the disulfide bond, it is necessary to further attach a solubilizing tag to an end of the target protein, such as TrxA tag (Zhao et al., 2016; Chinese patent CN 104755502 B), SUMO tag (Regina L. Bis et al., 2014), or to use a complex denaturation method (Y. Mohammed et al., 2012).

Therefore, there is still a need for low-cost, simple, and efficient methods for the production and purification of target polypeptides such as human growth hormone and interferon α2a.

### SUMMARY

Provided is a low-cost, simple, and efficient method for producing and purifying of a disulfide bond containing polypeptide based on a self-aggregating peptide and a cleavage tag.

In an aspect, provided is a fusion polypeptide, comprising a target polypeptide moiety and a self-aggregating peptide moiety, wherein the target polypeptide is a human growth hormone, wherein the target polypeptide moiety is linked to the self-aggregating peptide moiety via a spacer and wherein the cleavage tag comprises a cleavage site. In some embodiments, the fusion polypeptide may form an active aggregate via the self-aggregating peptide moiety after expression in a host cell. In some embodiments, the target polypeptide moiety in the fusion polypeptide according to the present disclosure is located at N-terminus of the fusion polypeptide. In other embodiments, the target polypeptide in the fusion polypeptide according to the present disclosure is located at C-terminus of the fusion polypeptide.

In some embodiments, the self-aggregating peptide moiety in the fusion polypeptide according to the present disclosure comprises an amphipathic self-assembling short peptide. In some embodiments, the self-aggregating peptide moiety comprises one or more tandem repeated amphipathic self-assembling short peptides.

In some embodiments, the amphipathic self-assembling short peptide in the fusion polypeptide according to the present disclosure is selected from the group consisting of an amphipathic β sheet short peptide, an amphipathic α helix short peptide and a surfactant-like short peptide. In some embodiments, a surfactant-like short peptide is preferable.

In some embodiments, the surfactant-like short peptide has 7-30 amino acid residues and has an amino acid sequence as shown in the following formula, from N-terminus to C-terminus:

A-B or B-A

wherein A is a peptide consisting of hydrophilic amino acid residues, the hydrophilic amino acid residues can be identical or different and are selected from the group consisting of Lys, Asp, Arg, Glu, His, Ser, Thr, Asn and Gln; B is a peptide consisting of hydrophobic amino acid residues, the hydrophobic amino acid residues can be identical or different and are selected from the group consisting of Leu, Gly, Ala, Val, Ile, Phe and Trp; A and B are linked via a peptide bond; and wherein the proportion of the hydrophobic amino acid residues in the surfactant-like short peptide is 55%-95%. In some embodiments, the surfactant-like short peptide has 8 amino acid residues, wherein the proportion of the hydrophobic amino acid residues in the surfactant-like short peptide is 75%. In some embodiments, the surfactant-like short peptide is selected from the group consisting of L6KD, L6KK, L6DD, L6DK, L6K2, L7KD and DKL6. In some embodiments, the surfactant-like short peptide in the fusion polypeptide according to the present disclosure is L6KD, of which the amino acid sequence is shown in SEQ ID NO: 1.

In some embodiments, the amphipathic β sheet short peptide has a length of 4-30 amino acid residues and the content of the hydrophobic amino acid residues is 40%-80%. In some embodiments, the amphipathic β sheet short peptide in the fusion polypeptide according to the present disclosure is EFK8, of which the amino acid sequence is shown in SEQ ID NO: 2.

In some embodiments, the amphipathic self-assembling short peptide is an amphipathic α helix short peptide having a length of 4-30 amino acid residues, and the content of the hydrophobic amino acid residues is 40%-80%. In some embodiments, the amphipathic α helix short peptide in the fusion polypeptide according to the present disclosure is α3-peptide, of which the amino acid sequence is shown in SEQ ID NO: 3.

In some embodiments, the target polypeptide in the fusion polypeptide according to the present disclosure is a human growth hormone. In some embodiments, the human growth hormone moiety comprises an amino acid sequence as shown in SEQ ID NO:5.

In some embodiments, in the fusion polypeptide according to the present disclosure, the spacer is directly linked to the target polypeptide moiety and/or the self-aggregating peptide moiety. In other embodiments, the spacer further comprises a linker at its N-terminus and/or C-terminus and is linked to the target polypeptide moiety and/or the self-aggregating peptide moiety via the linker.

In some embodiments, the cleavage site in the fusion polypeptide according to the present disclosure is selected from the group consisting of a temperature dependent cleavage site, a pH dependent cleavage site, an ion dependent cleavage site, an enzyme cleavage site or a self-cleavage site. In some embodiments, the cleavage site is a self-cleavage site. In some specific embodiments, the spacer is an intein, which comprise a self-cleavage site. In some embodiments, the intein is linked to the N-terminus or C-terminus of the human growth hormone moiety. In some embodiments, the intein is Mxe GyrA, which has a sequence as shown in SEQ ID NO: 4. In some alternative embodiments, the Mxe GyrA is linked to the C-terminus of the human growth hormone moiety.

In some embodiments, the linker in the spacer according to the present disclosure is a GS type linker, of which the amino acid sequence is shown in SEQ ID NO:6. In other embodiments, the linker is a PT type linker, of which the amino acid sequence is shown in SEQ ID NO:7.

In another aspect, provided is an isolated polynucleotide comprising a nucleotide sequence encoding the fusion polypeptide according to the present disclosure or a complementary sequence thereof.

In another aspect, provided is an expression construct, comprising the polynucleotide according to the present disclosure.

In another aspect, provided is a host cell, comprising the polynucleotide according to the present disclosure, or transformed with the expression construct according to the present disclosure, wherein the host cell is able to express the fusion polypeptide.

In some embodiments, the host cell is selected from the group consisting of a prokaryote cell, a yeast cell and a higher eukaryotic cell. In some specific embodiments, the prokaryote comprises bacteria of Escherichia genus, Bacillus genus, Salmonella genus, Pseudomonas genus and Streptomyces genus. More specifically, the prokaryote belongs to Escherichia genus, preferably, is E. coli.

In another aspect, provided is method for producing and purifying a human growth hormone, comprising the steps of:

(a) culturing the host cell according to the present disclosure, thereby expressing the fusion polypeptide according to the present disclosure;

(b) lysing the host cell, removing the soluble fraction of the cell lysate and recovering the insoluble fraction;

(c) releasing the soluble human growth hormone from the insoluble fraction via cleavage of the cleavage site; and

(d) removing the insoluble fraction in step (c) and recovering the soluble fraction containing the human growth hormone.

In some embodiments, the lysis is performed by sonication, homogenization, high pressure, hypotonicity, lyase, organic solvent or a combination thereof. In other embodiments, the lysis is performed under a weak alkaline pH condition. In some specific embodiments, the cleavage is a dithiothreitol (DTT) mediated self-cleavage.

In another aspect, provided is a fusion polypeptide, comprising a target polypeptide moiety and a self-aggregating peptide moiety, wherein the target polypeptide moiety is linked to the self-aggregating peptide moiety via a spacer and wherein the cleavage tag comprises a cleavage site. In some embodiments, the fusion polypeptide, after expression in the host cell, can form an active aggregate through the self-aggregating peptide moiety. In some embodiments, the target polypeptide in the fusion polypeptide according to the present disclosure is a human growth hormone or a human interferon α2a. In some embodiments, the target polypeptide moiety in the fusion polypeptide according to the present disclosure is located at N-terminus of the fusion polypeptide. In other embodiments, the target polypeptide moiety in the fusion polypeptide according to the present disclosure is located at C-terminus of the fusion polypeptide.

In some embodiments, the self-aggregating peptide moiety in the fusion polypeptide according to the present disclosure comprises an amphipathic self-assembling short peptide. In some embodiments, the self-aggregating peptide moiety comprises one or more tandem repeated amphipathic self-assembling short peptides.

In some embodiments, the amphipathic self-assembling short peptide in the fusion polypeptide according to the present disclosure is selected from the group consisting of an amphipathic β sheet short peptide, an amphipathic α helix short peptide and a surfactant-like short peptide. In some embodiments, the surfactant-like short peptide is preferable.

In some embodiments, the surfactant-like short peptide has 7-30 amino acid residues and has an amino acid sequence as shown in the following formula, from N-terminus to C-terminus:

A-B or B-A

wherein A is a peptide consisting of hydrophilic amino acid residues, the hydrophilic amino acid residues can be identical or different and are selected from the group consisting of ys, Asp, Arg, Glu, His, Ser, Thr, Asn and Gln; B is a peptide consisting of hydrophobic amino acid residues, the hydrophobic amino acid residues can be identical or different and are selected from the group consisting of Leu, Gly, Ala, Val, Ile, Phe and Trp; A and B are linked via a peptide bond; and wherein the proportion of the hydrophobic amino acid residues in the surfactant-like short peptide is 55%-95%. In some embodiments, the surfactant-like short peptide has 8 amino acid residues, and the proportion of the hydrophobic amino acid residues in the surfactant-like short peptide is 75%. In some embodiments, the surfactant-like short peptide is selected from the group consisting of L6KD, L6KK, L6DD, L6DK, L6K2, L7KD and DKL6. In some embodiments, the surfactant-like short peptide in the fusion polypeptide according to the present disclosure is L6KD, of which the amino acid sequence is shown in SEQ ID NO: 1.

In some embodiments, the amphipathic β sheet short peptide has a length of 4-30 amino acid residues; and the content of the hydrophobic amino acid residues therein is 40%-80%. In some embodiments, the amphipathic β sheet short peptide in the fusion polypeptide according to the present disclosure is EFK8, of which the amino acid sequence is shown in SEQ ID NO: 2.

In some embodiments, the amphipathic self-assembling short peptide is an amphipathic α helix short peptide having a length of 4-30 amino acid residues; and wherein the content of the hydrophobic amino acid residues therein is 40%-80%. In some embodiments, the amphipathic α helix short peptide in the fusion polypeptide according to the present disclosure is β3-peptide, of which the amino acid sequence is shown in SEQ ID NO: 3.

In some embodiments, the amphipathic self-assembling short peptide is an a triple helix peptide. In some embodiments, the a triple helix peptide in the fusion polypeptide according to the present disclosure is TZ1H, of which the amino acid sequence is shown in SEQ ID NO: 36.

In some embodiments, the target polypeptide in the fusion polypeptide according to the present disclosure comprises at least two thiol groups, for example, two thiol groups, three thiol groups, four thiol groups or more thiol groups, wherein a disulfide bond can be formed between the thiol groups. In some embodiments, the target polypeptide in the fusion polypeptide according to the present disclosure comprises one or more disulfide bonds. In some embodiments, the target polypeptide in the fusion polypeptide according to the present disclosure comprises one or more intramolecular disulfide bonds, for example, one disulfide bond, two disulfide bonds or more disulfide bonds.

In some embodiments, the target polypeptide in the fusion polypeptide according to the present disclosure has a length of 20-400 amino acids, for example, 30-300 amino acids, 35-250 amino acids, 40-200 amino acids.

In some embodiments, the target polypeptide in the fusion polypeptide according to the present disclosure is a human growth hormone. In some embodiments, the human growth hormone moiety comprises an amino acid sequence as shown in SEQ ID NO:5.

In some embodiments, the target polypeptide in the fusion polypeptide according to the present disclosure is a human interferon α2a. In some embodiments, the human interferon α2a moiety comprise an amino acid sequence as shown in SEQ ID NO:26.

In some embodiments, in the fusion polypeptide according to the present disclosure, the spacer is directly linked to the target polypeptide moiety and/or the self-aggregating peptide moiety. In other embodiments, the spacer further comprises a linker at its N-terminus and/or C-terminus and is linked to the target polypeptide moiety and/or the self-aggregating peptide moiety via the linker.

In some embodiments, the cleavage site in the fusion polypeptide according to the present disclosure is selected from the group consisting of a temperature dependent cleavage site, a pH dependent cleavage site, an ion dependent cleavage site, an enzyme cleavage site or a self-cleavage site. In some embodiments, the cleavage site is a self-cleavage site. In some specific embodiments, the spacer is an intein, which comprises a self-cleavage site. In some embodiments, the intein is linked to the N-terminus or C-terminus of the target polypeptide moiety. In some embodiments, the intein is linked to the C-terminus of the target polypeptide moiety. In some embodiments, the intein is Mxe GyrA, which has a sequence as shown in SEQ ID NO: 4. In some alternative embodiments, the Mxe GyrA is linked to the C-terminus of the human growth hormone moiety.

In some embodiments, the cleavage site in the fusion polypeptide according to the present disclosure is selected from the group consisting of a temperature dependent cleavage site, a pH dependent cleavage site, an ion dependent cleavage site, an enzyme cleavage site or a self-cleavage site. In some embodiments, the cleavage site is a pH dependent cleavage site. In some specific embodiments, the spacer is intein, which comprises a pH dependent cleavage site. In some embodiments, the intein is linked to the N-terminus or C-terminus of the target polypeptide moiety. In some embodiments, the intein is linked to the N-terminus of the target polypeptide moiety. In some embodiments, the intein is Mtu ΔI-CM, which has a sequence as shown in SEQ ID NO: 27. In some alternative embodiments, the Mtu ΔI-CM is linked to N-terminus of the human growth hormone moiety. In some alternative embodiments, the Mtu ΔI-CM is linked to N-terminus of the human interferon α2a moiety.

In some embodiments, the Mtu ΔI-CM comprises a pH dependent cleavage site, which is cleaved under an acidic condition, preferably cleaved under a weak acidic condition, for example, cleaved under a condition of pH 6.0-6.5, preferably cleaved under a condition of pH 6.2. In some embodiments, the pH dependent cleavage site is not cleaved under an alkaline condition.

In some embodiments, the intein is a mutant of Mtu ΔI-CM. In some embodiments, the Mtu ΔI-CM has a mutation at position 73 and/or position 430. In some embodiments, the mutation at position 73 in the mutant of Mtu ΔI-CM is H73Y or H73V. In some embodiments, the mutation at position 430 in the mutant of Mtu ΔI-CM is T430V, T430S or T430C. In some specific embodiments, the amino acid sequence of the mutant of Mtu ΔI-CM having H73Y and T430V is shown in SEQ ID NO: 28. In some specific embodiments, the amino acid sequence of the mutant of Mtu ΔI-CM having H73V and T430S is shown in SEQ ID NO: 29. In some specific embodiments, the amino acid sequence of the mutant of Mtu ΔI-CM having H73V and T430C is shown in SEQ ID NO: 30.

In some embodiments, the linker in the spacer according to the present disclosure is a GS type linker, of which the amino acid sequence is shown in SEQ ID NO:6. In other embodiments, the linker is a PT type linker, of which the amino acid sequence is shown in SEQ ID NO:7.

In yet another aspect, provided is an isolated polynucleotide comprising a nucleotide sequence encoding the fusion polypeptide according to the present disclosure or a complementary sequence thereof.

In yet another aspect, provided is an expression construct, comprising the polynucleotide according to the present disclosure.

In yet another aspect, provided is a host cell, comprising the polynucleotide according to present disclosure, or transformed with the expression construct according to present disclosure, wherein the host cell is able to express the fusion polypeptide.

In some embodiments, the host cell is selected from the group consisting of a prokaryote cell, a yeast cell and a higher eukaryotic cell. In some specific embodiments, the prokaryote comprises a bacteria of Escherichia genus, Bacillus genus, Salmonella genus, Pseudomonas genus, and Streptomyces genus. More specifically, the prokaryote belongs to Escherichia genus, preferably, is E. coli.

In yet another aspect, provided is a method for producing and purifying a target polypeptide, comprising the steps of:

(a) culturing the host cell according to the present disclosure, thereby expressing the fusion polypeptide the present disclosure;

(b) lysing the host cell, removing the soluble fraction of the cell lysate and recovering the insoluble fraction;

(c) releasing the soluble target polypeptide from the insoluble fraction via cleavage of the cleavage site; and

(d) removing the insoluble fraction in step (c) and recovering the soluble fraction containing the target polypeptide.

In some embodiments, the lysis is performed by sonication, homogenization, high pressure, hypotonicity, lyase, organic solvent or a combination thereof. In other embodiments, the lysis is performed under a weak alkaline pH condition. In some specific embodiments, the cleavage is a pH dependent cleavage, for example, cleaved under an acidic condition, preferably cleaved under a weak acidic condition, for example, cleaved under a condition of pH 6.0-6.5, preferably cleaved under a condition of pH 6.2.

### BRIEF DESCRIPTION OF THE DRAWINGS

FIG. 1 shows the expression and purification strategy of human growth hormone hGH based on the self-aggregating peptide and the structure of the expression vector. A: Expression and purification strategy; B: Structure of vectors pET30-hGH-Mxe-L6KD, pET30-hGH-Mxe-EFK8, pET30-hGH-Mxe-α3.

FIG. 2 shows the results of SDS-PAGE analysis of the expression and purification of human growth hormone hGH fusion protein. A: Based on L6KD self-aggregating peptide; B: Based on EFK8 self-aggregating peptide; C: Based on α3-peptide self-aggregating peptide.

FIG. 3 shows the mass spectrometry of human growth hormone hGH.

FIG. 4 shows the biological activity analysis of human growth hormone hGH.

FIG. 5 shows the expression and purification strategy of human growth hormone hGH and human interferon α2a based on self-aggregating peptide and the structure of the expression vector. A: Expression and purification strategy; B: Structure of vectors pET32-L6KD-Mtu ΔI-CM-hGH, pET32-L6KD-Mtu ΔI-CM mutant 1-hGH, pET32-L6KD-Mtu ΔI-CM mutant 2-hGH, pET32-L6KD-Mtu ΔI-CM mutant 3-hGH, pET32-ELK16-Mtu ΔI-CM mutant 2-hGH, pET32-EFK8-Mtu ΔI-CM mutant 2-hGH, pET32-α3-Mtu ΔI-CM mutant 2-hGH, pET32-TZ1H-Mtu ΔI-CM mutant 2-hGH, pET32-L6KD-Mtu ΔI-CM-IFNα2a, pET32-L6KD-Mtu ΔI-CM mutant 1-IFNα2a, pET32-L6KD-Mtu ΔI-CM mutant 2-IFNα2a, pET32-L6KD-Mtu ΔI-CM mutant 3-IFNα2a.

FIG. 6 shows the results of SDS-PAGE analysis of the expression and purification of human growth hormone hGH fusion protein. A: The expression and purification results of LB medium from different Mtu ΔI-CM mutants; B: The expression and purification results of fermentation medium from different Mtu ΔI-CM mutants; C: The supernatants after cleavage of fusion proteins with different self-aggregating peptides expressed in LB medium.

FIG. 7 shows the results of SDS-PAGE analysis column purification of human growth hormone hGH.

FIG. 8 shows the RP-HPLC analysis of human growth hormone hGH.

FIG. 9 shows the MS analysis of human growth hormone hGH.

FIG. 10 shows the Native-PAGE analysis of human growth hormone hGH.

FIG. 11 shows the CD (Circular Dichroism) analysis of human growth hormone hGH.

FIG. 12 shows the results of SDS-PAGE analysis of the expression and purification of human interferon α2a fusion protein. A: Mtu ΔI-CM; B: Mtu ΔI-CM mutant 1 and 2; C: Mtu ΔI-CM mutant 3; D: Expression and purification results of Mtu ΔI-CM mutant 2 in fermentation medium.

### DETAILED DESCRIPTION

The present disclosure is not limited to the specific methods, protocols, reagents, etc. described herein as they may vary. The terminology used herein is for the purpose of describing specific embodiments only rather than limiting the scope. Unless otherwise defined, all technical and scientific terms used herein have the same meanings as commonly understood by those skilled in the art.

In an aspect, provided is a fusion polypeptide, comprising a target polypeptide moiety and a self-aggregating peptide moiety, wherein the target polypeptide is a human growth hormone, wherein the target polypeptide moiety is linked to the self-aggregating peptide moiety via a spacer and wherein the cleavage tag comprises a cleavage site.

In another aspect, provided is a fusion polypeptide, comprising a target polypeptide moiety and a self-aggregating peptide moiety, wherein the target polypeptide moiety is linked to the self-aggregating peptide moiety via a spacer and wherein the cleavage tag comprises a cleavage site.

In yet another aspect, provided is a fusion polypeptide, comprising a target polypeptide moiety and a self-aggregating peptide moiety, wherein the target polypeptide is a human interferon α2a, wherein the target polypeptide moiety is linked to the self-aggregating peptide moiety via a spacer and wherein the cleavage tag comprises a cleavage site.

In another aspect, provided is an isolated polynucleotide comprising a nucleotide sequence encoding the fusion polypeptide according to the present disclosure or a complementary sequence thereof.

In yet another aspect, provided is an expression construct, comprising the polynucleotide according to the present disclosure.

In yet another aspect, provided is a host cell, comprising the polynucleotide according to present disclosure, or transformed with the expression construct according to present disclosure, wherein the host cell is able to express the fusion polypeptide.

In yet another aspect, provided is a method for producing and purifying a human growth hormone, comprising the steps of: (a) culturing the host cell according to the present disclosure, thereby expressing the human fusion polypeptide the present disclosure; (b) lysing the host cell, removing the soluble fraction of the cell lysate and recovering the insoluble fraction; (c) releasing the soluble human growth hormone from the insoluble fraction via cleavage of the cleavage site; and (d) removing the insoluble fraction in step (c) and recovering the soluble fraction containing the human growth hormone.

In yet another aspect, provided is a method for producing and purifying a target polypeptide, comprising the steps of: (a) culturing the host cell according to the present disclosure, thereby expressing the fusion polypeptide the present disclosure; (b) lysing the host cell, removing the soluble fraction of the cell lysate and recovering the insoluble fraction; (c) releasing the soluble target polypeptide from the insoluble fraction via cleavage of the cleavage site; and (d) removing the insoluble fraction in step (c) and recovering the soluble fraction containing the target polypeptide.

As used herein, the terms “peptide”, “polypeptide” and “protein” are used interchangeably and are defined as biomolecules consisting of amino acid residues linked by peptide bonds.

As used herein, the amino acid sequence of the “target polypeptide” according to the present disclosure contains at least two cysteines, such as two cysteines, three cysteines, four cysteines or more cysteines, and the cysteines can form an intramolecular disulfide bond, such as one intramolecular disulfide bond, two intramolecular disulfide bonds or more intramolecular disulfide bonds. The “target polypeptide” according to the present disclosure contains at least two thiol groups, such as two thiol groups, three thiol groups, four thiol groups or more thiol groups, and a disulfide bond can be formed between the thiol groups, such as one intramolecular disulfide bond, two intramolecular disulfide bonds or more intramolecular disulfide bonds. The target polypeptide can have a length of 20-400 amino acids, such as 30-300 amino acids, 35-250 amino acids, 40-200 amino acids.

As used herein, “human growth hormone” and “target polypeptide” are used interchangeably and refer to a protein hormone secreted by the anterior pituitary gland, of which the mature form is an aglycosylated hydrophilic globulin with the signal peptide been removed, consists of 191 amino acids, has two disulfide bonds, and has a relative molecular weight of about 22 kDa. The human growth hormone moiety of the fusion polypeptide according to the present disclosure comprises an amino acid sequence shown in SEQ ID NO:5.

As used herein, “human interferon α2a” and “target polypeptide” are used interchangeably, which is a multifunctional and highly active inducible protein produced by leukocytes and lymphocytes, consists of 165 amino acids, contains two pairs of intramolecular disulfide bonds, and has a relative molecular weight of about 19.2 kDa. The human interferon α2a moiety of the fusion polypeptide according to the present disclosure comprises an amino acid sequence shown in SEQ ID NO:26.

In some specific embodiments, the “target polypeptide” according to the present disclosure has a structure similar to “human growth hormone”. In some specific embodiments, the “target polypeptide” according to the present disclosure has a structure similar to “human interferon α2a”.

In some embodiments, the fusion polypeptide, after expression in the host cell, can form an active aggregate through the self-aggregating peptide moiety. In some embodiments, the target polypeptide moiety in the fusion polypeptide according to the present disclosure is located at N-terminus of the fusion polypeptide. In other embodiments, the target polypeptide in the fusion polypeptide according to the present disclosure is located at C-terminus of the fusion polypeptide.

As used herein, “self-aggregating peptide” refers to a polypeptide fused to the target polypeptide moiety and capable of mediating the formation of an insoluble active aggregate by the fusion polypeptide in the host cell after expression therein.

As used herein, “active aggregate” means that the human growth hormone moiety is still able to fold correctly and remain active or that the human growth hormone moiety in the aggregate can be in a soluble form after separation from the self-aggregating peptide.

Without intention to be bound by any theory, some amphipathic (amphipathic) polypeptides are known in the art to spontaneously form specific self-assembled structures due to hydrophobic interactions and other driving forces and due to their separate hydrophilic and hydrophobic domains (Zhao et al., 2008). The inventors have surprisingly found that some amphipathic short peptides with self-assembly ability can induce the formation of intracellular active aggregates. The amphipathic self-assembling short peptide used as the self-aggregating peptide according to the present disclosure can be selected from the group consisting of an amphipathic β sheet short peptide, an amphipathic α helix short peptide and a surfactant-like short peptide. The amphipathic self-assembling short peptide used as the self-aggregating peptide according to the present disclosure can also be selected from the group consisting of an a triple helix peptide.

As used herein, “surfactant-like peptide” is a class of amphipathic polypeptides which can be used as the self-aggregating peptide according to the present disclosure, which generally consist of 7-30 amino acid residues, extends about 2-5 nm in length, is structurally similar to a lipid, and is composed of a hydrophobic amino acid tail and a hydrophilic amino acid head. The properties of a surfactant-like structure are similar to those of surfactants, and they can form assembled structures such as micelles and nanotubes in aqueous solutions. Surfactant-like short peptide suitable for use as self-aggregating peptide according to the present disclosure can has a length of 7-30 amino acid residues, and has an amino acid sequence as shown in the following formula, from N-terminus to C-terminus:

A-B or B-A,

wherein A and B are linked via a peptide bond. A is a hydrophilic head consisting of hydrophilic amino acids, the hydrophilic amino acid residues can be identical or different polar amino acids and are selected from the group consisting of Lys, Asp, Arg, Glu, His, Ser, Thr, Asn and Gln. Examples of A comprise KD, KK or DK etc. B is a hydrophobic tail consisting of hydrophobic amino acid residues, the hydrophobic amino acid residues can be identical or different non-polar amino acids and are selected from the group consisting of Leu, Gly, Ala, Val, Ile, Phe and Trp. Examples of B comprise LLLLLL(L6), L7 or GAVIL etc. The proportion of hydrophobic amino acids in the surfactant-like short peptide according to the present disclosure is higher than that of hydrophilic amino acids, and the proportion of hydrophobic amino acids in the surfactant-like short peptide can be 55-95%, 60-95%, 65-95%, 70-95%, 75-95%, 80-95%, 85-95%, 90-95%. In some embodiments, the surfactant-like short peptide has 8 amino acid residues, wherein the proportion of hydrophobic amino acids is 75%. In an aqueous solution, the surfactant-like peptide self-assembles such that the hydrophobic tails are aggregated inside, and the hydrophilic heads are exposed to the solution to interact with the aqueous solution, preventing the hydrophobic region from contacting the aqueous solution. Specific examples of surfactant-like short peptides suitable for the self-aggregating peptide according to the present disclosure include L6KD, L6KK, L6DD, L6DK, L6K2, L7KD and DKL6 etc. The fusion polypeptide according to the present disclosure uses L6KD, of which the amino acid sequence is shown in SEQ ID NO: 1.

In addition, it is known to those skilled in the art that surfactant-like peptides with the above-mentioned structures (such as L6KD, L6K2, L6D2, etc.) have similar activities and can mediate fusion proteins to form insoluble active aggregates in cells (Zhou et al., 2012).

As used herein, “amphipathic β sheet short peptide” refers to a short peptide with 4-30 amino acid residues, which is composed of alternating arrangements of hydrophobic amino acids and charged hydrophilic amino acids. When forming a P sheet, hydrophobic amino acid residues are located at one side, alternating positively and negatively charged hydrophilic amino acid residues are located at the other side. These short peptides can form self-assembled structures under hydrophobic interactions, electrostatic interactions and hydrogen bonding. In general, the longer the length of the amphipathic β-sheet structure or the stronger the hydrophobicity, the easier the self-assembly occurs and the stronger the mechanical strength of the formed self-aggregates. In order to ensure sufficient self-assembly ability, the amphipathic β sheet short peptide according to the present disclosure should contain a certain amount of hydrophobic amino acids. The amphipathic β sheet short peptide according to the present disclosure comprises 40-80%, 45-70%, 50-60%, e.g., about 50% of hydrophobic amino acid residues. A specific example of amphipathic P sheet short peptide used as the self-aggregating peptide according to the present disclosure is EFK8, of which the amino acid sequence is shown in SEQ ID NO: 2.

α helix is a protein secondary structure in which the peptide chain backbone extends in a helical manner around an axis. As used herein, “amphipathic α helix short peptide” refers to a peptide with 4-30 amino acid residues, which has a unique arrangement of hydrophilic, hydrophobic amino acids compared to an ordinary α helix, such that in the formed α helix structure, hydrophilic amino acids are mainly located at one side and hydrophobic amino acids are mainly located at the other side. It is speculated that amphipathic α-helix achieves self-assembly in an aqueous solution through the formation of coiled-coils, wherein two α-helix bind through hydrophobic interaction and further stabilize such binding through electrostatic interactions of charged amino acids. The amphipathic α helix short peptide according to the present disclosure comprises 40-80%, 45-70%, 50-60%, e.g., about 50% of hydrophobic amino acid residues. A specific example of amphipathic α helix short peptide used as the self-aggregating peptide according to the present disclosure is α3-peptide, of which the amino acid sequence is shown in SEQ ID NO: 3. As used herein, “a triple helix peptide” consists of six heptapeptide repeats with three histidine residues at the d-position of the first, third and fifth heptapeptide repeats. A specific example of a triple helix peptide used as the self-aggregating peptide according to the present disclosure is TZ1H, e, of which the amino acid sequence is shown in SEQ ID NO: 36 (Lou et al., 2019).

It has been reported in the field that a polypeptide with self-aggregating property is formed by tandem repeating multiple repeating units, such as elastin-like ELP, which consists of 110 VPGXG repeating units, and its aggregation property is associated with the number of repeating units (Banki, et al., 2005; MacEwan and Chilkoti, 2010). It has also been reported that the self-aggregation tendency of an amphipathic β-sheet composed of multiple repeating units increases with number of the repeating units (Zhang et al., 1992). It can be expected that a polypeptide composed of multiple “amphipathic self-assembling short peptides” in tandem can retain or even acquire enhanced self-assembling ability.

Accordingly, the self-aggregating peptide moiety according to the present disclosure can comprise one or more of the amphipathic self-assembling short peptides in series. The self-aggregating peptide moiety according to the present disclosure can comprise 1-150, 1-130, 1-110, 1-90, 1-70, 1-50, 1-30, 1-10, 1-5, for example 1, 2, 3, 4, 5 of the amphipathic self-assembling short peptides. Two or more amphipathic self-assembling short peptide in the self-aggregating peptide moiety can form a tandem repeat. In order to facilitate recombinant manipulation and take into account production cost, it is desirable to use less repeats. Therefore, in some embodiments, the “self-aggregating peptide moiety” comprises only one amphipathic self-assembling short peptide.

In addition, it has been reported that some protein domains, such as β-amyloid peptide, VP1, MalE31, CBD_{clos} or the like can also induce fusion proteins to form aggregates, and it is expected herein that such domains can also be used as the “self-aggregating peptide” according to the present disclosure. However, the structures of these domains are relatively complex and the mechanism by which they induce aggregation remains unclear (Mitraki, 2010). amphipathic self-assembling short peptides with relatively simple structure and short length are preferably used in the present disclosure.

It has been found in current studies that after expression of a self-aggregating peptide (such as an amphipathic self-assembling peptide) with the ability to induce formation of an active aggregate and a target polypeptide as a fusion protein in a host cell, the expressed fusion protein can form an insoluble aggregate. The formation of an aggregate can avoid degradation of the fusion protein by intracellular protease, thereby increasing the yield of the target polypeptide. After cell lysis, the insoluble aggregate can be simply collected from cell lysates by centrifugal precipitation or filtration to remove soluble impurities and achieve preliminary purification of the fusion protein. Then, by cleaving the cleavage site in the linker between the self-aggregating peptide moiety and the target polypeptide, the soluble fraction containing the target polypeptide is released from the insoluble fraction (precipitate) and distributed in the supernatant, where the insoluble impurities can be removed by simple centrifugal precipitation or filtration and the soluble target polypeptide can be collected. The production of a polypeptide by such a self-aggregating peptide-based method can simplify the separation and purification steps, avoid the expensive purification columns, and significantly reduce the production cost.

It is also reported in the prior art that the above method is only suitable for producing a class of proteins without a disulfide bond, such as Bacillus subtilis lipase A (LipA) (Van Pouderoyen et al., 2001), Aspergillus fumigatus Type II ketamine oxidase (AMA) (Collard et al., 2008), Bacillus pumilus xylosidase (XynB) (the structure information can be found in the Protein Data Bank PDB, https://www.rcsb.org/structure/1YIF) or the like. A target protein with disulfide bonds (such as CCL5 (2 disulfide bonds), SDF-1α (3 disulfide bonds), and leptin (1 disulfide bond) tend to form an aggregate after intein-mediated cleavage and cannot be released into in the supernatant. The reason these cleaved target proteins remain aggregated may be due to exposed hydrophobic sequences or difficulty in forming correct disulfide bonds in the periplasmic space of E. coli (Zhao et al., 2016). In order to solve the problems caused by disulfide bonds, current studies have found that a protein with disulfide bonds can be efficiently produced by adding a solubilizing tag to one end of the target protein (Zhao et al., 2016; CN 104755502 B), such as TrxA tag (Zhao et al., 2016), SUMO tag (Regina L. Bis et al., 2014).

However, the present inventors unexpectedly found that although a human growth hormone has two disulfide bonds, it can be efficiently produced by the above-described method utilizing the self-aggregating peptide without the addition of a solubilizing tag. In addition, the present inventors also found that a human interferon-α2a having a structure similar to human growth hormone with two disulfide bonds can also be produced by the above-mentioned method using the self-aggregating peptide.

As used herein, “spacer” refers to a polypeptide composed of amino acids with a certain length, which includes a sequence required to achieve cleavage, such as protease recognition sequences for enzymatic cleavage, intein sequences for self-cleavage, or the like, to connect each part of the fusion protein without affecting the structure and activity of each part. Therefore, the spacer according to the present disclosure comprises a “cleavage site”. In the fusion polypeptide according to the present disclosure, the spacer is directly linked to the target polypeptide moiety and/or the self-aggregating peptide moiety. In other embodiments, the spacer further comprises a linker at its N-terminus and/or C-terminus, which is linked to the target polypeptide moiety and/or the self-aggregating peptide moiety via the linker.

In some specific embodiments, the spacer is an intein, comprising a self-cleavage site. In some embodiments, the intein is linked to N-terminus or C-terminus of the human growth hormone moiety. It should be understood that those skilled in the art can select the appropriate intein according to the needs and select the appropriate connection position of the intein.

The cleavage site used for releasing the soluble target polypeptide moiety from the insoluble fraction (precipitate) according to the present disclosure can be selected from the group consisting of a temperature dependent cleavage site, a pH dependent cleavage site, an ion dependent cleavage site, an enzyme cleavage site or a self-cleavage site, or any other cleavage site known to those skilled in the art. The preferable cleavage site in the present disclosure is capable of self-cleavage, for example, comprising an amino acid sequence of a self-cleavable intein. This is due to the reason that an intein-based cleavage method does not require the addition of an enzyme or the use of a harmful substance such as hydrogen bromide used in chemical methods, but simply induces cleavage by changing the buffer environment where the aggregates are located (Wu et al., 1998; TELENTI et al., 1997). A variety of self-cleaving inteins are known in the art, such as a series of inteins with different self-cleaving properties from NEB. In some embodiments, the cleavage site can also be a pH-dependent cleavage site.

In some specific embodiments according to the present disclosure, the intein is Mxe GyrA, having a sequence of SEQ ID NO: 4. In some alternative embodiments, the Mxe GyrA is linked to C-terminus of the human growth hormone moiety. In a specific embodiment according to the present disclosure, the intein Mxe GyrA can induce self-cleavage of the intein at its amino terminus by adding an appropriate amount of dithiothreitol (DTT) to the buffer system. Those skilled in the art can determine the DTT concentration and reaction time as needed. Optionally, DTT is removed in subsequent operations.

In some specific embodiments according to the present disclosure, the intein is Mtu ΔI-CM, having a sequence of SEQ ID NO: 27. In some alternative embodiments, the Mtu ΔI-CM is linked at N-terminus of the human growth hormone moiety. In some alternative embodiments, the Mtu ΔI-CM is linked at N-terminus of the human interferon α2a moiety. In a specific embodiment, the intein Mtu ΔI-CM can induce self-cleavage of the intein at its carboxyl terminus by a buffer system at pH 6.2.

As used herein, “Mtu ΔI-CM” is derived from Mtu recA wildtype intein, which retains 110 amino acids of N-terminus and 58 amino acids of C-terminus by deleting the endonuclease domain of Mtu recA extra-large intein to obtain a very small intein, and then introduce four mutations: C1A, V67L, D24G, D422G (Wood et al., 1999).

The present disclosure also provides Mtu ΔI-CM mutants, and these mutants can also be used as the intein according to the present disclosure. In some specific embodiments, as Mtu ΔI-CM comprises a pH-dependent cleavage site, before the final in vitro cleavage step, it is possible that the self-cleavage may occur during in vivo expression due to insufficient pH control, resulting in loss of part of the target polypeptide, so as to give in vivo self-cleavage in premature maturation. In order to reduce the proportion of premature self-cleavage in vivo, the mutation(s) at position 73 and/or position 430 of Mtu ΔI-CM are introduced. Alternatively, the mutation at position 73 is selected from the group consisting of H73Y and H73V, the mutation at position 430 is selected from the group consisting of T430V, T430S and T430C. Preferably, the mutant has a mutation combination selected from the group consisting of: H73Y/T430V (SEQ ID NO: 28), H73V/T430S (SEQ ID NO: 29) and H73V/T430C (SEQ ID NO: 30). More preferably, the mutant has a mutation combination selected from the group consisting of: H73V/T430S (SEQ ID NO: 29) and H73V/T430C (SEQ ID NO: 30). In addition, as the activity of Mtu ΔI-CM is sensitive to temperature, the in vivo self-cleavage phenomenon of premature maturation can also be suppressed by lowering the temperature. For example, reducing the temperature to 18° C. when expressing the fusion protein, and cooling the strains sufficiently before adding IPTG to induce the expression of the recombinant protein will reduce the proportion of self-cleavage in vivo.

Those skilled in the art will understand that, in order to reduce the mutual interference between different parts in the fusion protein according to the present disclosure, different parts of the fusion protein can be connected by a linker. As used herein, “linker” refers to a polypeptide with a certain length composed of amino acids with low hydrophobic and low charge effects, which can fully unfold the connected parts when used in a fusion protein and make them fully fold into their respective native conformations.

The linkers commonly used in the art include, for example, a flexible GS type linker rich in glycine (G) and serine (S); a rigid PT type linker rich in proline (P) and threonine (T). In some embodiments, the amino acid sequence of the GS type linker used in the present disclosure is shown in SEQ ID NO:6. In other embodiments, the amino acid sequence of the PT type linker used in the present disclosure is shown in SEQ ID NO:7.

In the production of a polypeptide drug, it is often required that the recombinant polypeptide has the same sequence as the target polypeptide, that is, there is no additional amino acid residue at both ends, so that the produced polypeptide has the same pharmacokinetics as the naturally occurring polypeptide. In the present disclosure, it can be achieved by choosing an appropriate cleavage site and the way it is linked to the target polypeptide. It is clear to those skilled in the art to make such a selection according to the feature of the cleavage site. For example, in a specific embodiment, the Mxe GyrA of the cleavage site can be directly linked to C-terminus of the target polypeptide moiety, such that it is directly linked to C-terminus of the target polypeptide moiety and thereby there is no additional amino acid residue between the human growth hormone moiety. In other specific embodiments, between the “target polypeptide” and “spacer” according to the present disclosure there is a short sequence which improves the cleavage efficiency, such as “MRM”, without affecting the final activity of the target polypeptide. In some other specific embodiments, the amino acid sequence of the target polypeptide obtained by the self-cleavage of the carboxyl-terminal of Mtu ΔI-CM is completely consistent with that of the target sequence, which is significant for polypeptide drugs, either from the point of view of drug approval or biological effect. It will be understood by those skilled in the art that when spacers with different cleavage sites are selected, cleavage can be performed to generate a target polypeptide without redundant amino acid residues at the C-terminus and/or N-terminus.

As mentioned above, provided is also a polynucleotide comprising the nucleotide sequence encoding the fusion polypeptide according to the present disclosure or a complementary sequence thereof. As used herein, “polynucleotide” refers to a macromolecule in which multiple nucleotides are linked by 3′-5′-phosphodiester bonds, wherein the nucleotides comprise ribonucleotides and deoxyribonucleotides. The sequence of the polynucleotide according to the present disclosure can be codon-optimized for different host cells (such as E. coli), thereby improving the expression of the fusion protein. Methods for codon optimization are known in the art.

As mentioned above, provided is also an expression construct comprising the above-described polynucleotide according to the present disclosure. In the expression construct according to the present disclosure, the sequence of the polynucleotide encoding the fusion protein is operably linked to the expression control sequence to perform the desired transcription and finally produce the fusion polypeptide in the host cell. Suitable expression control sequence includes but not limited to a promoter, an enhancer, a ribosomal interaction sites such as ribosome binding site, a polyadenylation site, a transcriptional splicing sequence, a transcriptional termination sequence, a mRNA-stabilizing sequence or the like.

Vector used in the expression construct according to the present disclosure includes that can replicate autonomously in the host cell, such as plasmid vector; and that can be integrated and replicate with host cell DNA. Many commercially available suitable vectors are suitable. In a specific embodiment, the expression construct according to the present disclosure is derived from pET30a(+) of Novagen.

Provided is also a host cell comprising the polynucleotide according to the present disclosure or is transformed by the expression construct according to the present disclosure, wherein the host cell is able to express the fusion polypeptide according to the present disclosure. The host cells used to express the fusion polypeptide according to the present disclosure comprises a prokaryote cell, a yeast cell and a higher eukaryotic cell. Exemplary prokaryote cell comprises a bacteria of Escherichia genus, Bacillus genus, Salmonella genus, Pseudomonas genus, and Streptomyces genus. In a preferable embodiment, the host cell is a cell of Escherichia genus, preferably E. coli. In a specific embodiment according to the present disclosure, the host cell used is a cell of strain E. coli BL21(DE3) (Novagen).

The recombinant expression construct according to the present disclosure can be introduced into the host cell by any of well-known techniques including but not limited to heat shock transformation, electroporation, DEAE-dextran transfection, microinjection, liposome mediated transfection, calcium phosphate precipitation, protoplast fusion, particle bombardment, viral transformation or the like.

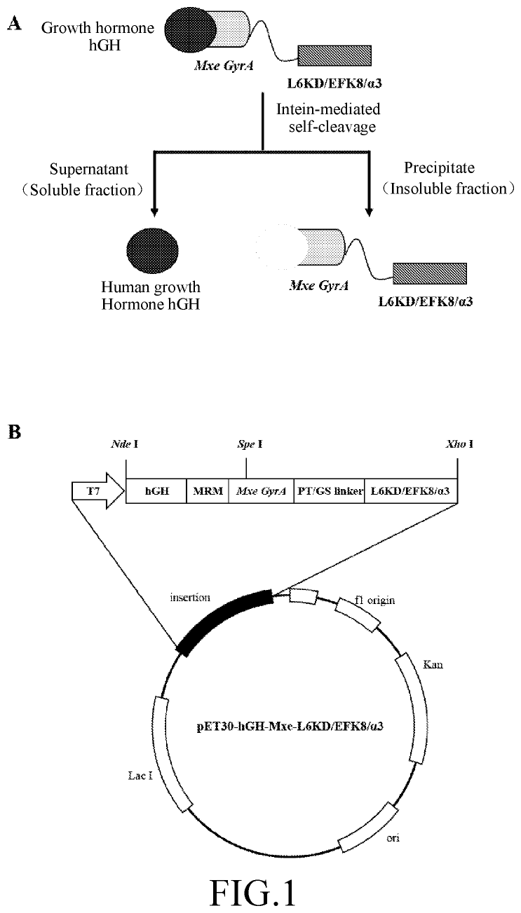

Provided is also a method for producing and purifying a human growth hormone, comprising the steps of: (a) culturing the host cell according to the present disclosure, thereby expressing the fusion polypeptide according to the present disclosure; (b) lysing the host cell, removing the soluble fraction of the cell lysate and recovering the insoluble fraction; (c) releasing the soluble human growth hormone from the insoluble fraction via cleavage of the cleavage site; and (d) removing the insoluble fraction in step (c) and recovering the soluble fraction containing the human growth hormone. A schematic diagram of the method of the present invention can be seen in FIG. 1A.

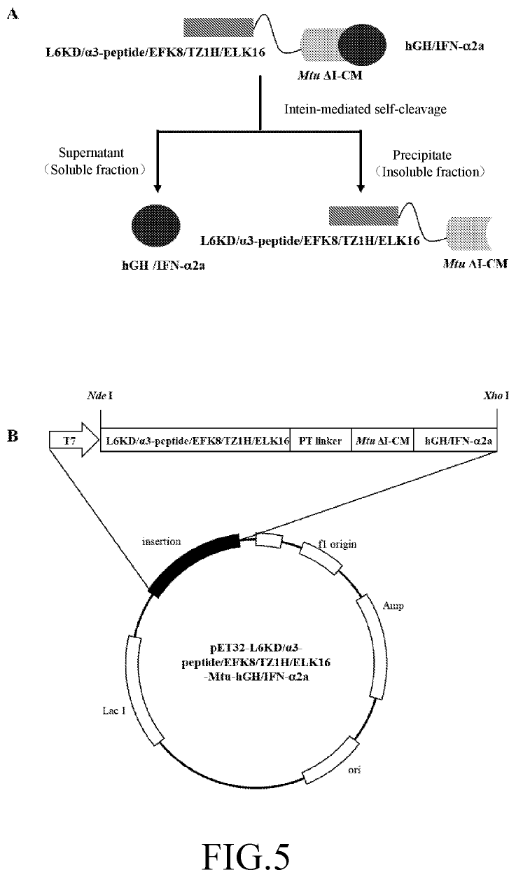

Provided is also a method for producing and purifying a target polypeptide, comprising the steps of: (a) culturing the host cell according to the present disclosure, thereby expressing the fusion polypeptide according to the present disclosure; (b) lysing the host cell, removing the soluble fraction of the cell lysate and recovering the insoluble fraction; (c) releasing the soluble target polypeptide from the insoluble fraction via cleavage of the cleavage site; and (d) removing the insoluble fraction in step (c) and recovering the soluble fraction containing the target polypeptide. A schematic diagram of the method of the present invention can be seen in FIG. 5A.

In the present disclosure, the method for lysing the host cell is selected from the group consisting of treating methods commonly used in the art, such as ultrasound, homogenization, high pressure (e.g., in a French press), hypotonicity (osmolysis), detergent, lyase, organic solvent or a combination thereof, and the lysis is carried out under a weak alkaline pH condition (e.g., pH 7.5-8.5), thereby allowing the cell membranes of the host cell to be lysed, such that the active aggregates are released from the cell but remain insoluble.

The released aggregates are directly recovered in the form of precipitation, omitting the step of obtaining the fusion protein in the form of precipitation by changing environmental conditions (such as temperature, ion concentration, pH value, etc.), and avoid the effects of the acutely changed environmental conditions on stability and activity.

In the conventional production of growth hormone, as human growth hormone has a disulfide bond, it is necessary to secrete the growth hormone into the periplasmic space of E. coli to solve the problem of expression caused by the disulfide bonds. The expression by the protein secretion into the periplasmic space is generally considered to be at the level of 0.1-10 mg/L, mostly at the level of about 1 mg/L, and the following two methods are mainly used in the purification process: purification using a very expensive antibody against growth hormone (antibody-specific purification, but the antibody is very expensive, and can be used for less batches, that is, after a few batches, new antibody has to be used) packed column for purification is used (Chang et al, 1986); or an affinity tag is used, and then the fusion protein is purified by a series of complex steps: 1) purification of the fusion protein with the affinity tag, 2) changing the buffer, 3) adding a protease to cleave the tag, 4) affinity tag purification to remove the protease and the tag, 5) changing the buffer again, etc., and then, molecular sieve purification is performed to obtain the growth hormone (Nguyen et al, 2014; Moony et al, 2014).

In contrary to the addition of a solubilizing tag to overcome the problem of disulfide bonds as taught in the prior art, although the target polypeptide human growth hormone according to the present disclosure has two disulfide bonds, the present inventors have surprisingly found that the fusion method based on the self-aggregating peptide without adding a solubilizing tag can also successfully produce large amounts of active human growth hormone. The self-aggregating peptide used in the present disclosure can induce the fusion protein to form a large number of active protein aggregates, avoid the degradation of the human growth hormone in the host, and is beneficial for correct folding in the prokaryotic cell to form the active human growth hormone. The human growth hormone obtained in the present disclosure is a correctly folded soluble protein, which does not require tedious denaturation and renaturation operations in the protocols and has high yield and purity. The purification of the human growth hormone according to the present disclosure requires low level of equipment, does not need a purification column, and has a low production cost and easy operation.

As used herein, “purity” refers to the purity of the target protein, that is, the ratio of the target polypeptide such as human growth hormone to the total protein in the purified solution. As the target protein is expressed through cells, there are a large number of other proteins in the cells (e.g., thousands of proteins in E. coli), it has always been a key technical challenge to purify the target protein from such a large variety of protein mixtures. Through the steps of cell disruption, centrifugation, and separation after cleavage, there are substantially only proteins and inorganic salts in the purified solution. Therefore, the higher the proportion of human growth hormone in the purified solution, the higher the purity of the production is.

### EXAMPLES

To make the technical solutions and advantages according to the present disclosure clearer, the embodiments of the present disclosure will be described in more details below by Examples. It should be understood that the Examples should not be construed as limiting and those skilled in the art can use the principles according to the present disclosure to make further adjustments.

The methods used in the following Examples are conventional unless otherwise specified, and the specific steps can be referred to, for example, Molecular Cloning: A Laboratory Manual (Sambrook, J., Russell, David W., Molecular Cloning: A Laboratory Manual, 3rd edition, 2001, NY, Cold Spring Harbor). The primers used were all synthesized by Shanghai Sangon Biotechnology Co., Ltd.

### Example 1: Construction of Expression Construct for Human Growth Hormone Fusion Protein Containing Intein Mxe GyrA

The construction processes of the expression vectors pET30-hGH-Mxe-L6KD, pET30-hGH-Mxe-EFK8, pET30-hGH-Mxe-α3 used in the Examples were similar and the construction of pET30-hGH-Mxe-L6KD was used as an example. The required primers were designed by oligo 6 and synthesized by Shanghai Sangon Biotechnology Co., Ltd., and were shown in Table 1.

TABLE 1
Oligonucleotide primers used in this Example

| | | SEQ |
|---|---|---|
| | | ID |
| Primer | Nucleotide sequence ^{a} | NO |
| hGH-F | 5′-CGCCATATGTTCCCGACCATCC | 8 |
| | CGCTG-3′ | |
| hGH-R | 5′-GATGCACATTCGCATGAAACCG | 9 |
| | CAAG-3′ | |
| Mxe-L6KD-F | 5′-CCTAATGTTTCATGCGAATGTGC | 10 |
| | ATCACG-3′ | |
| Mxe-L6KD-R | 5′-TGCTCGAGTCAATCTTTCAGCAGCAG | 11 |
| | CAGCAGCAGCGGCGTCGGGGTTGG-3′ | |
| Mxe-EFK8-R | 5′-TGCTCGAGTCACTTGAACTCGA | 12 |
| | ATTCGAACTCGAACGGC | |
| | GTCGGGGT-3′ | |
| hGHalpha-R | 5′-CGGACTAGTGCATCTCCCGTGAT | 13 |
| | GCACATTCGCATGAAACCG-3′ | |

^{a} The underlined parts of the primers were the recognition sites of the restriction enzymes Nde I, Xho I and Spe I, respectively.

First, the polynucleotide sequence of human growth hormone hGH was obtained from NCBI (NCBI No: AAA98618.1), the codons were optimized for E. coli with jcat software, and the gene fragment was obtained through gene synthesis by Shanghai Sangon Biotechnology Co., Ltd. The growth hormone hGH polynucleotide fragment was obtained by PCR amplification using the synthesized gene as template and hGH-F and hGH-R as primers. The Q5 polymerase from NEB (New England Biolab (NEB)) was used in the PCR reaction, and the PCR conditions were: 98° C. 30 sec; 98° C. 10 sec, 60° C. 30 sec, 72° C. 30 sec, with 30 cycles in total; finally, 72° C. for 2 min. After the reaction was completed, the PCR amplification products were separated and recovered with 1% agarose gel.

Using pET30-lipA-Mxe-L6KD (Xing Lei et al., 2011) as template, MxeL6KD-F and MxeL6KD-R as primers, the Mxe-L6KD polynucleotide fragment was amplified by PCR reaction. Q5 polymerase from NEB was used in the PCR reaction, and the PCR conditions were: 98° C. 30 sec; 98° C. 10 sec, 60° C. 30 sec, 72° C. 30 sec, 30 cycles in total; finally, 72° C. 2 min. After the reaction was completed, the PCR amplification products were separated and recovered with 1% agarose gel. Then the two fragments hGH and Mxe-L6KD were subjected to overlapping PCR reaction: firstly without adding primers: 98° C. 30 sec; 98° C. 10 sec, 68° C. 30 sec, 72° C. 25 sec, 15 cycles in total; finally 72° C. 5 min. Then, primers hGH-F and MxeL6KD-R were added, 98° C. 30 sec; 98° C. 10 sec, 68° C. 30 sec, 72° C. 25 sec, 30 cycles in total; finally, 72° C. 5 min. After the reaction was completed, the PCR amplification products were detected by electrophoresis, and the results showed that the correct bands as expected were amplified by PCR, and then, were separated and recovered. The overlapping PCR products were double-digested with restriction enzymes Nde I and Xho I, and then, ligated with T4 ligase to the plasmid pET30(a) double-digested by the same enzymes, and the ligated products were transformed into E. coli DH5α competent cells. The transformed cells were spread on a LB plate supplemented with 50 μg/mL kanamycin to screen for positive clones, and the plasmids were extracted and sequenced. The sequencing results showed that the sequence of pET30-hGH-Mxe-L6KD as cloned was correct.

The sequenced and correct plasmids were then transformed into E. coli BL21(DE3) (Novagen) competent cells, and the transformed cells were spread on a LB plate supplemented with 50 μg/mL kanamycin to select positive clones for subsequent expression and purification. Similar methods were used to obtain pET30-hGH-Mxe-EFK8 and pET30-hGH-Mxe-α3 plasmids and their expression strains, respectively. When pET30-hGH-Mxe-EFK8 was constructed, primer Mxe-EFK-R was used for cloning instead of Mxe-L6KD-R; when pET30-hGH-Mxe-α3 was constructed, primer hGH-F and hGHalpha-R were used for cloning from pET30-hGH-Mxe-L6KD to obtain hGH-Mxe nucleotide fragment, which was then inserted into pET30-lipA-Mxe-α3 plasmid vector double-digested by Nde I and Spe I restriction enzymes (Lin Zhanglin et al., 2018). The structures of the constructed pET30-hGH-Mxe-L6KD, pET30-hGH-Mxe-EFK8, pET30-hGH-Mxe-α3 plasmids were shown in FIG. 1B.

### Example 2: Expression and Purification of Human Growth Hormone Fusion Protein

The strains constructed in Example 1 (containing plasmid pET30-hGH-Mxe-L6KD, pET30-hGH-Mxe-EFK8, pET30-hGH-Mxe-α3) were inoculated into LB liquid medium containing 50 μg/mL kanamycin and were cultured in a shaker at 37° C. to logarithmic phase (OD₆₀₀=0.4-0.6). IPTG was added to 0.2 mM, induction was performed at 18° C. for 18 h and 30° C. for 6 h, the cells were harvested, and the bacterial concentration OD₆₀₀ was measured (the amount of cells with OD₆₀₀ of 1 in 1 mL was referred to as 1 OD below).

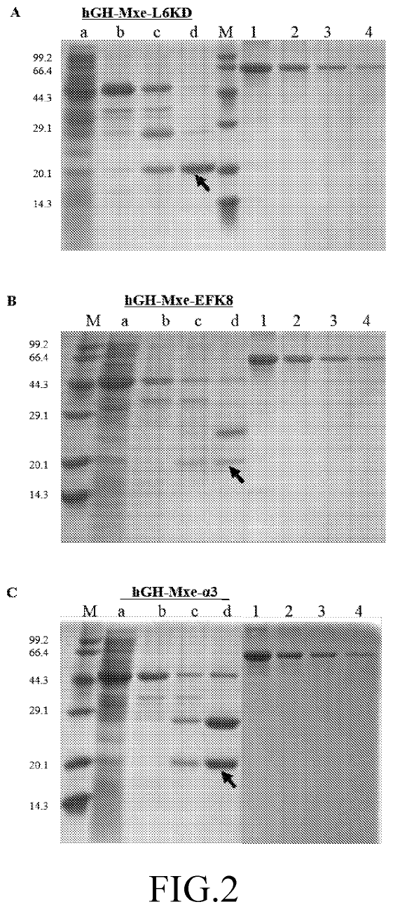

The cells were resuspended to 20 OD/mL in lysis buffer B1 (2.4 g Tris, 29.22 g NaCl, 0.37 g Na₂EDTA.2H₂O dissolved in 800 mL of water, adjusted to pH 8.5, and made up to 1 L with water), ultrasonic fragmentation (fragmentation conditions: power 200 W, ultrasonic time 3 sec, interval time 3 sec, ultrasonic times 99) was performed. Centrifugation was performed at 4° C., 12,000 rpm for 20 min, and the supernatants and pellets were collected, respectively. The pellets were washed twice with lysis buffer, then fully resuspended with cleavage buffer (20 mM Tris-HCl, 500 mM NaCl, 40 mM dithiothreitol, 1 mM EDTA, pH 8.5) and placed at 4° C. overnight for 24 h, allowing the intein fully self-cleaved. The suspension was then centrifuged, and the resulting supernatants and pellets were determined with SDS-PAGE together with the pre-cleaved pellets (the pellets were resuspended in the same volume of lysis buffer as in the previous resuspension step). The result was shown in FIG. 2. Lanes a-d were the expression and purification samples of human growth hormone hGH, respectively a: cell lysate supernatants; b: cell lysate pellets, where clear aggregates of fusion protein expression can be detected; c: pellets separated after cleavage; d: supernatants isolated after cleavage, where a clear band of human growth hormone hGH can be detected. Lane 1-4 was the protein quantification standard containing bovine serum protein BSA, and the loading amounts were 4 μg, 2 μg, 1 μg, and 0.5 μg.

According to the protein quantitative standard, the target band was analyzed by densitometric analysis using Bio-Rad Quantity ONE gel quantitative analysis software, and the aggregate yield formed by the fusion protein, the yield of human growth hormone hGH released into the supernatants after intein-mediated self-cleavage, the cleavage efficiency of Mxe GyrA, the recovery rate of human growth hormone hGH and the purity in the supernatants could be calculated and were shown in Table 2.

TABLE 2
Expression and purification of human growth hormone hGH

| | Aggregate | Human growth | | | |
|---|---|---|---|---|---|
| | expression ^{a} | hormone hGH | | | Human growth |
| | (μg/mg wet | yield ^{b} (μg/mg | cleavage | Recovery | hormone hGH |
| Fusion protein | cell weight) | wet cell weight) | efficiency ^{c} | rate ^{d} (%) | purity (%) |
| hGH-Mxe-L6KD | 150.0 | 21.4 | 61.3 | 30.2 | 88.2 |
| hGH-Mxe-EFK8 | 44.9 | 2.8 | 64.2 | 13.4 | 31.4 |
| hGH-Mxe-α3 | 119.8 | 19.8 | 52.8 | 35.1 | 46.7 |

^{a} yield of protein aggregate,
^{b} yield of human growth hormone hGH after intein-mediated self-cleavage (when the bacterial concentration at OD₆₀₀ was 2, the E. coli cells in LB medium produced 2.66 mg wet cell weight per liter),
^{c} self-cleavage efficiency mediated by intein = 100% × (the amount of expressed aggregate before cleavage − the amount of aggregate remained after cleavage)/yield of the aggregate before cleavage,
^{d} recovery rate = 100% × hGH real yield/theoretical yield of human growth hormone hGH produced by protein aggregate with complete cleavage.

The three fusion proteins as used (hGH-Mxe-L6KD, hGH-Mxe-EFK8, hGH-Mxe-α3) all existed in the form of precipitate, and the aggregate expression was 44.9-150.0 μg/mg wet cell weight. Three fusion proteins were self-cleaved by intein Mxe GyrA, hGH was separated from Mxe-L6KD/EFK8/α3-peptide, the cleavage efficiency was 52.8-64.2%, and the yield of human growth hormone hGH released into the supernatants after cleavage was 2.8-21.4 μg/mg wet cell weight, the hGH purity recovered after cleavage was 31.4-88.2%. hGH-Mxe-L6KD fusion protein showed the highest yield and purity of human growth hormone hGH, that is, the yield of human growth hormone hGH which was obtained by one-step purification through the present purification technology based on self-aggregating peptide and self-cleavage tag was 21.4 μg/mg wet cell weight, and the purity was 88.2%.

### Example 3: Molecular Weight Determination of Human Growth Hormone hGH

Taking the human growth hormone hGH sample obtained from L6KD self-aggregating peptide in Experimental Example 2 as an example, the molecular weight was determined. The human growth hormone hGH sample was dialyzed with mobile phase (solution A: solution B=1:1) to prepare 2 mg/mL hGH sample, and the molecular weight was analyzed by HPLC-MS. Instrument: Agilent 1260 HPLC connected to Waters SYNAPT G2-S time-of-flight mass spectrometry system; Chromatographic column: Acquity UPLC BEH C18 column (2.1 mmx 100 mm, 1.7 μm particle size, 130 Å, Waters, USA); Mobile phase: solution A 0.1% (v/v) aqueous formic acid, solution B 0.1% (v/v) formic acid in acetonitrile, the gradient used was shown in Table 2; the injection volume was 10 μL, the flow rate was 0.4 mL/min, and the temperature was 60° C.

TABLE 3
Parameters for setting the mobile phase gradient

| Time | Ratio of solution | Ratio of solution |
|---|---|---|
| (min) | A (% (v/v)) | B (% (v/v)) |
| 0 | 75 | 25 |
| 50 | 30 | 70 |
| 55 | 15 | 85 |
| 65 | 15 | 85 |

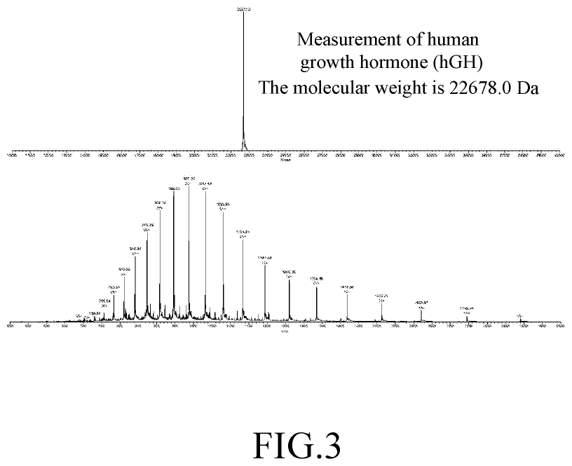

It can be seen from FIG. 3 that the obtained molecular weight was 22,678.0 Da, which was substantially consistent with the calculated molecular weight of 22,678.8 Da, and the difference was within the range of machine measurement error by 0.8 Da, proving that the obtained hGH sequence was correct.

### Example 4: Detection of the Biological Activity of Human Growth Hormone

Taking the human growth hormone hGH sample obtained from L6KD self-aggregating peptide in Example 2 as an example, the biological activity was determined. The proliferation testing cell NB2-11 cell line (European Collection of Authenticated Cell Cultures (ECACC)), a standard of human growth hormone, was used as the testing cell. The NB2-11 cells in good growth condition were trypsinized and counted. Serum-free medium was used to resuspend the cells to prepare a cell suspension, and 5,000 cells per well were inoculated into a 96-well cell culture plate for 24 h for serum starvation. Each sample was diluted to the set concentration, added into the corresponding cell culture well, and incubated in the incubator for 24 h. Proliferation assay was performed using CCK8 kit (Shanghai Beyotime Biotechnology Co., Ltd.). 20 μL of CCK8 solution was added to each well; the culture plate was incubated in an incubator for 2 h; the absorbance at 450 nm was measured with a microplate reader. The detecting samples included bovine serum albumin (BSA), human growth hormone hGH obtained from L6KD self-aggregating peptide in Example 2, commercial human growth hormone hGH (proteintech, USA), and the sample concentrations were 1, 5, 10, 20, 30, 40, 50 ng/mL.

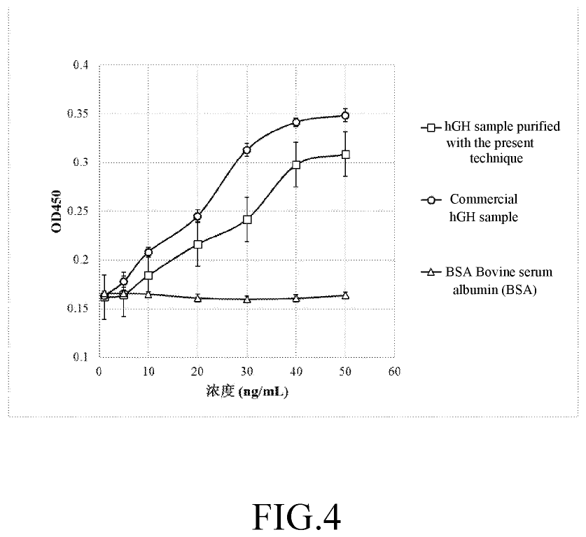

As shown in FIG. 4, the human growth hormone hGH purified by the present method can effectively promote the proliferation of NB2-11 cells, which increased with the increase of the added concentrations from 1 to 50 ng/mL, and the trend was basically the same as that of commercial hGH samples. With addition of 50 ng/mL hGH, the proliferative activity of human growth hormone hGH purified by the present method on NB2-11 cells was 88.5% of that of the commercial hGH sample. Considering that the tested hGH sample purity was 88.2%, the biological activity of the obtained human growth hormone hGH sample was comparable to that of commercial human growth hormone hGH.

### Example 5: Construction of Expression Vector for Human Growth Hormone Fusion Protein Containing Intein Mtu ΔI-CM

The construction procedures of the expression vectors in the present Example were similar: pET32-L6KD-Mtu ΔI-CM-hGH, pET32-L6KD-Mtu ΔI-CM mutant 1-hGH, pET32-L6KD-Mtu ΔI-CM mutant 2-hGH, pET32-L6KD-Mtu ΔI-CM mutant 3-hGH, pET32-ELK16-Mtu ΔI-CM mutant 2-hGH, pET32-EFK8-Mtu ΔI-CM mutant 2-hGH, pET32-α3-Mtu ΔI-CM mutant 2-hGH, pET32-TZ1H-Mtu ΔI-CM mutant 2-hGH. Taking the construction of pET32-L6KD-Mtu ΔI-CM-hGH as an example, the required primers were designed by oligo 6 and synthesized by Shanghai Sangon Biotechnology Co., Ltd., and were shown in Table 4.

TABLE 4
Oligonucleotide primers used
in this Example

| | | SEQ |
|---|---|---|
| | | ID |
| Primer | Nucleotide sequence | NO |
| J20001-Mtu-F | 5′-CTGCTGCTGAAAGATCC′ | 14 |
| | AACCCC-3 | |
| J19042-Mtu-R | 5′-ATGGTCGGGAAGTTATGAACC | 15 |
| | ACAACGCCTT-3′ | |
| J19040-hGH-F | 5′-TTGTGGTTCATAACTTCCCGAC | 16 |
| | CATCCCGCTGTCTCGT-3′ | |
| J19041-hGH-R | 5′-TTAGCAGCCGGATCTCAGTGG | 17 |
| | T-3′ | |

J19040-hGH-F and J19041-hGH-R were used as primers, and the growth hormone hGH polynucleotide fragment was amplified by PCR reaction (PCR instrument (Bio-rad/C1000 Touch)). The Q5 polymerase from NEB (New England Biolab (NEB)) was used in the PCR reaction, and the PCR conditions were: 98° C. 30 sec; 98° C. 10 sec, 60° C. 30 sec, 72° C. 30 sec, cycles in total; finally, 72° C. 2 min. After the reaction was completed, the PCR amplification products were subjected to 1% agarose gel electrophoresis, and then recovered using an ultra-thin DNA gel product recovery kit (Magen, D2110-03).

J20001-Mtu-F and J19042-Mtu-R were used as primers, the L6KD-Mtu ΔI-CM nucleotide fragment was amplified from pET30a-L6KD-Mtu ΔI-CM-AMA by PCR reaction (Zhou B. et al., 2012). The Q5 polymerase from NEB (New England Biolab (NEB)) was used in the PCR reaction, and the PCR conditions were: 98° C. 30 sec; 98° C. 10 sec, 72° C. 30 sec, 72° C. 1 min, cycles in total; finally, 72° C. 2 min. After the reaction was completed, the PCR amplification products were separated and recovered with 1% agarose gel.

The growth hormone hGH polynucleotide fragment and L6KD-Mtu ΔI-CM nucleotide fragment were subjected to overlapping PCR reactions. The Q5 polymerase from NEB was used in the PCR reaction, and the PCR conditions were: 98° C. 30 sec, 98° C. 10 sec, 72° C. 30 sec, 72° C. 2 min, 30 cycles in total; finally, 72° C. 2 min. The PCR amplification products were subjected to 1% agarose gel electrophoresis, and then, recovered using an ultra-thin DNA gel product recovery kit (Magen, D2110-03). The purified fragment and pET32a plasmid (Novagen) were double-digested with restriction enzymes EcoR I and Xho I, respectively, and then, the corresponding fragments were recovered for purification, and then ligated with T4 DNA ligase after purification. The ligated products were transformed into E. coli DH5α competent cells and the transformed cells were spread on a LB plate supplemented with 100 μg/mL carbenicillin to screen for positive clones. The plasmids were extracted with a plasmid extraction kit and sequenced.

The sequenced and correct plasmids were then transformed into E. coli BL21(DE3) (Novagen) competent cells, and the transformed cells were spread on a LB plate supplemented with 100 μg/mL carbenicillin to screen positive clones for subsequent expression and purification.

Similar procedures were used to obtain pET32-L6KD-Mtu ΔI-CM mutant 1-hGH, pET32-L6KD-Mtu ΔI-CM mutant 2-hGH, pET32-L6KD-Mtu ΔI-CM mutant 3-hGH, pET32-ELK16-Mtu ΔI-CM mutant 2-hGH, pET32-EFK8-Mtu ΔI-CM mutant 2-hGH, pET32-α3-Mtu ΔI-CM mutant 2-hGH, pET32-TZ1H-Mtu ΔI-CM mutant 2-hGH plasmids and the expression strains thereof. The structure of the constructed pET32-L6KD-Mtu ΔI-CM-hGH plasmid was shown in FIG. 5B.

### Example 6: Expression and Purification of Human Growth Hormone Fusion Protein in LB Medium

The strains constructed in Example 5 (containing the respective plasmids as described above) were inoculated into LB liquid medium containing 100 μg/mL carbenicillin and cultured in a shaker at 37° C. to log phase (OD₆₀₀=0.4-0.6), a final concentration of 0.2 mM IPTG was added, and the induction was performed at 18° C. for 24 h. The cells were harvested and the bacterial concentration OD₆₀₀ was measured. The amount of cells with OD₆₀₀ of 1 in 1 mL was referred to as 1 OD below.

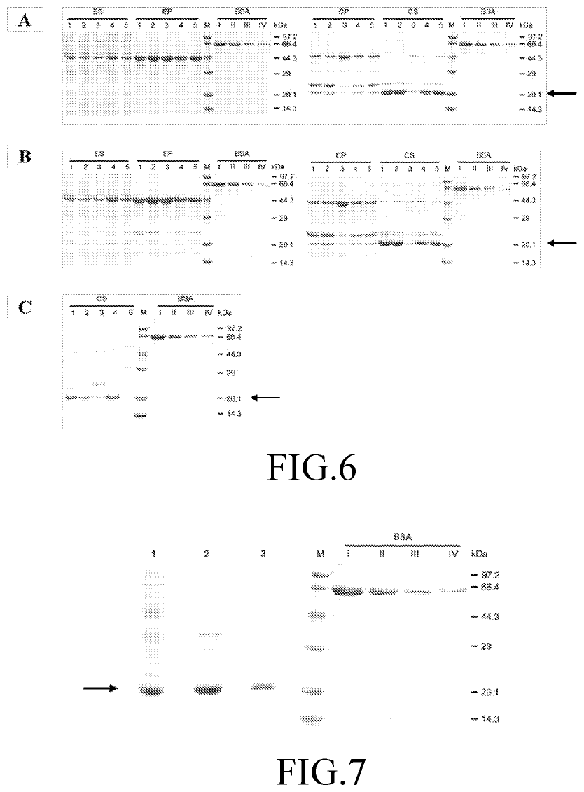

The cells were resuspended to 20 OD/mL in lysis buffer B1 (2.4 g Tris, 29.22 g NaCl, 0.37 g Na₂EDTA.2H₂O dissolved in 800 mL of water, adjusted to pH 8.5, and made up to 1 L with water), and ultrasonic fragmentation (fragmentation conditions: power 200 W, ultrasonic time 3 sec, interval time 3 sec, ultrasonic times 99) was performed. Centrifugation was performed at 4° C., 15,000 g for 20 min, and the supernatants and pellets were collected. The pellets were washed with an equal volume of lysis buffer twice, then fully resuspended with an equal volume of cleavage buffer (PBS supplemented with 40 mM Bis-Tris, pH 6.2, 2 mM EDTA) and placed at 25° C. for 24 h, allowing the intein fully self-cleaved. After centrifugation at 4° C., 15,000 g for 20 min, the pellets were resuspended with an equal volume of lysis buffer. The resulting supernatants and pellets were determined with SDS-PAGE together with the pre-cleaved pellets. The result was shown in FIG. 6A. Lanes ES, EP, CP, CS were human growth hormone hGH expression and purification samples, respectively. ES: cell lysate supernatants; EP: cell lysate pellets, where clear aggregates of fusion protein expression can be detected; CP: pellets separated after cleavage; CS: supernatants separated after cleavage, where a clear band for human growth hormone hGH can be detected; lanes 1-5 were Mtu ΔI-CM (without cooling at 18° C.), Mtu ΔI-CM (with cooling at 18° C.), Mtu ΔI-CM mutant 1, Mtu ΔI-CM mutant 2, Mtu ΔI-CM mutant 3; lanes I-IV were standards for protein quantification with the successive loading amounts of 2.5 μg, 1.25 μg, 0.625 μg, 0.3125 μg. The results of SDS-PAGE detection of different aggregated peptides were shown in FIG. 6C, a clear band for human growth factor hGH can be detected from the supernatant isolated after the cleavage of lane CS, lanes 1-5 were L6KD, ELK16, EFK8, 0.3, TZ1H.

According to the protein quantitative standard, the target band was analyzed by densitometric analysis using ImageJ gel quantitative analysis software, and the aggregate yield formed by the fusion protein, the yield of human growth hormone hGH released into the supernatants after intein-mediated self-cleavage, the cleavage efficiency of Mtu ΔI-CM, the recovery rate of human growth hormone hGH and the purity in the supernatants could be calculated and were shown in Table 5.

TABLE 5
Expression and purification of human growth hormone hGH

| | Aggregate | | | | |
|---|---|---|---|---|---|
| | expression ^{a} | Human growth | | | |
| | (mg/L | hormone hGH | | | Human growth |
| | culture | yield ^{b} (mg/L | Cleavage | Recovery | hormone hGH |
| Fusion protein | solution) | culture solution) | efficiency ^{c} | rate ^{d} (%) | purity (%) |
| L6KD-Mtu-hGH | 423 | 62 | 64 | 29 | 80 |
| (without cooling at | | | | | |
| 18° C.) | | | | | |
| L6KD-Mtu-hGH | 446 | 72 | 72 | 32 | 82 |
| (with cooling at | | | | | |
| 18° C.) | | | | | |
| I6KD-Mtu(1)- | 536 | 8 | 31 | 3 | 49 |
| hGH | | | | | |
| L6KD-Mtu(2)- | 472 | 50 | 61 | 21 | 77 |
| hGH | | | | | |
| L6KD-Mtu(3)- | 510 | 70 | 61 | 27 | 82 |
| hGH | | | | | |
| ELK16-Mtu(2)- | 33 | 4 | 42 | 24 | 98 |
| hGH | | | | | |
| EFK8-Mtu(2)- | 33 | 2 | 36 | 12 | 29 |
| hGH | | | | | |
| α3-Mtu(2)-hGH | 303 | 33 | 46 | 22 | 93 |
| TZ1H-Mtu(2)- | 4 | 1 | 22 | 50 | 17 |
| hGH | | | | | |

^{a} yield of protein aggregate,
^{b} yield of human growth hormone hGH after intein-mediated self-cleavage (amount of protein produced by E. coli cells per liter of LB medium),
^{c} self-cleavage efficiency mediated by intein = 100% × ( the amount of expressed aggregate before cleavage − the amount of aggregate remained after cleavage)/yield of the aggregate before cleavage,
^{d} recovery rate = 100% × hGH real yield/theoretical yield of human growth hormone hGH produced by protein aggregate with complete cleavage.

The four different Mtu ΔI-CM mutant fusion proteins as used (L6KD-Mtu-hGH, L6KD-Mtu(1)-hGH, L6KD-Mtu(2)-hGH, L6KD-Mtu(3)-hGH) and four different aggregating peptide fusion proteins (ELK16-Mtu ΔI-CM mutant 2-hGH., EFK8-Mtu ΔI-CM mutant 2-hGH., α3-Mtu ΔI-CM mutant 2-hGH., TZ1H-Mtu ΔI-CM mutant 2-hGH) all existed in the form of precipitate and the aggregate expression of four different Mtu ΔI-CM mutant 2 (Mtu(2)) was 446-536 mg/L LB culture solution. Four different Mtu ΔI-CM mutant 2 fusion proteins were subjected to intein Mtu ΔI-CM self-cleavage, hGH was separated from L6KD-Mtu, the cleavage efficiency was 31-72%, the yield of human growth hormone hGH released into the supernatants after cleavage was 8-72 mg/L LB culture solution, and the hGH purity recovered after cleavage was 49-82%. The L6KD-Mtu-hGH fusion protein showed the highest yield and purity of human growth hormone hGH, that is, with cooling at 18° C., through the present purification technology based on self-aggregating peptide and self-cleavage tag, the yield of human growth hormone hGH was 72 mg/L LB culture solution wet cell weight, and purity was 82%. The aggregate expression of four different aggregating peptides was 4-303 mg/L LB culture solution, the four different aggregating peptides were subjected to intein Mtu ΔI-CM self-cleavage, hGH was separated from L6KD-Mtu, the cleavage efficiency was 22-46%, the yield of human growth hormone hGH released into the supernatants after cleavage was 1-33 mg/L LB culture solution, and the hGH purity recovered after cleavage was 17-98%.

### Example 7: Expression and Purification of Human Growth Hormone Fusion Protein in Fermentation Medium

The strains constructed in Example 5 were inoculated into fermentation medium containing 100 μg/mL carbenicillin (Shao-Yang Hu et al., 2004), and cultured in a shaker at 37° C. to log phase (OD₆₀₀=0.4-0.6). A final concentration of 0.2 mM IPTG was added, induction was performed at 18° C. for 24 h, the cells were harvested, and the bacterial concentration OD₆₀₀ was measured. The amount of cells with OD₆₀₀ of 1 in 1 mL was referred to as 1 OD below. The fermentation medium components used were shown in Table 6.

TABLE 6
Components of fermentation medium

| Components | Concentration | Components | Concentration |
|---|---|---|---|
| (NH₄)₂SO₄ | 5 g L⁻¹ | Glucose | 20 g L⁻¹ |
| Yeast extract | 20 g L⁻¹ | KH₂PO₄ | 6.75 g L⁻¹ |
| Na₂HPO₄•12H₂O | 3 g L⁻¹ | citric acid | 3 g L⁻1 |
| MgCl₂ | 1.5 g L⁻¹ | NH₄Cl | 0.1 g L⁻¹ |
| tryptone | 30 g L⁻¹ | Trace metal | 6 ml L⁻¹ |
| | | solution | |

Trace metal solution (dissolved with 3M HCl), 6 mL
Trace metal solution were added per 1 L medium

| | | | |
|---|---|---|---|
| FeSO₄•7H₂O | 10 mg | ZnSO₄•7H₂O | 2.25 mg |
| CaCl₂•2H₂O | 1.35 mg | MnSO₄•5H₂O | 0.5 mg |
| CuSO₄•5H₂O | 1 mg | AlCl•6H₂O | 0.3 mg |
| (NH₄)₆Mo₇O₂₄•4H₂O | 0.1 mg | H₃BO₃ | 0.2 mg |
| Thiamin HCl | 2 mg | | |

Glucose and other components were sterilized separately, sterilized at 121° C. for 20 min, and the trace element solution was filtered and sterilized on an ultra-clean workbench with a 0.22 μm filter. After the medium was prepared, carbenicillin with a final concentration of 100 mg/L was added prior to use.

The cells were resuspended to 20 OD/mL in lysis buffer B1 (2.4 g Tris, 29.22 g NaCl, 0.37 g Na₂EDTA.2H₂O dissolved in 800 mL of water, adjusted to pH 8.5, and made up to 1 L with water), and ultrasonic fragmentation (fragmentation conditions: power 200 W, ultrasonic time 3 sec, interval time 3 sec, ultrasonic times 99) was performed. After centrifugation at 4° C., 15,000 g for 20 min, the pellets were resuspended with an equal volume of lysis buffer. The resulting supernatants and pellets were determined with SDS-PAGE together with the pre-cleaved pellets. The result was shown in FIG. 6B. Lanes ES, EP, CP, CS were human growth hormone hGH expression and purification samples, respectively. ES: cell lysate supernatants; EP: cell lysate pellets, where clear aggregates of fusion protein expression can be detected; CP: pellets separated after cleavage; CS: supernatants separated after cleavage, where a clear band for human growth hormone hGH can be detected; lanes 1-5 were Mtu ΔI-CM (without cooling at 18° C.), Mtu ΔI-CM (with cooling at 18° C.), Mtu ΔI-CM mutant 1, Mtu ΔI-CM mutant 2, Mtu ΔI-CM mutant 3. Lanes I-IV was the protein quantification standard containing bovine serum albumin BSA with the successive loading amounts of 2.5 μg, 1.25 μg, 0.625 μg, 0.3125 g.

According to the protein quantitative standard, the target band was analyzed by densitometric analysis using ImageJ gel quantitative analysis software, and the aggregate yield formed by the fusion protein, the yield of human growth hormone hGH released into the supernatants after intein-mediated self-cleavage, the cleavage efficiency of Mtu ΔI-CM, the recovery rate of human growth hormone hGH and the purity in the supernatants could be calculated and were shown in Table 7.

TABLE 7
Expression and purification of human growth hormone hGH

| | Aggregate | | | | |
|---|---|---|---|---|---|
| | expression ^{a} | Human growth | | | |
| | (mg/L | hormone hGH | | | Human growth |
| | culture | yield ^{b} (mg/L | Cleavage | Recovery | hormone hGH |
| Fusion protein | solution) | culture solution) | efficiency ^{c} | rate ^{d} (%) | purity (%) |
| L6KD-Mtu-hGH | 1696 | 333 | 62 | 39 | 86 |
| (without cooling at | | | | | |
| 18° C.) | | | | | |
| L6KD-Mtu-hGH | 1737 | 311 | 63 | 36 | 88 |
| (with cooling at | | | | | |
| 18° C.) | | | | | |
| L6KD-Mtu(1)- | 2983 | 69 | 29 | 5 | 56 |
| hGH | | | | | |
| L6KD-Mtu(2)- | 2124 | 292 | 52 | 27 | 76 |
| hGH | | | | | |
| L6KD-Mtu(3)- | 2018 | 362 | 47 | 36 | 79 |
| hGH | | | | | |

^{a} yield of protein aggregate,
^{b} yield of human growth hormone hGH after intein-mediated self-cleavage (amount of protein produced by E. coli cells per liter of LB medium),
^{c} self-cleavage efficiency mediated by intein = 100% × ( the amount of expressed aggregate before cleavage − the amount of aggregate remained after cleavage)/yield of the aggregate before cleavage,
^{d} recovery rate = 100% × hGH real yield/theoretical yield of human growth hormone hGH produced by protein aggregate with complete cleavage.

The four different Mtu ΔI-CM fusion proteins as used (L6KD-Mtu-hGH, L6KD-Mtu(1)-hGH, L6KD-Mtu(2)-hGH, L6KD-Mtu(3)-hGH) all existed in the form of precipitate and the aggregate expression of four different Mtu ΔI-CM was 696-2,983 mg/L fermentation culture solution. Four different Mtu ΔI-CM fusion proteins were subjected to intein Mtu ΔI-CM self-cleavage, hGH was separated from L6KD-Mtu, the cleavage efficiency was 29-63%, the yield of human growth hormone hGH released into the supernatants after cleavage was 69-362 mg/L fermentation culture solution, and the hGH purity recovered after cleavage was 56-88%.

### Example 8: Fine Purification of Human Growth Hormone Fusion Protein

Taking the human growth hormone hGH sample obtained from L6KD self-aggregating peptide in Example 6 as an example, about 12 mg of human growth hormone hGH sample obtained from L6KD self-aggregating peptide was subjected to an anion exchange column (Capto HiRes Q 5/50) and a molecular sieve column (Sephacryl S200HR (16/60)) for fine purification. During ion exchange column purification, the unbound proteins was washed with binding buffer (20 mM Tris-HCl, pH 8.0) after loading, then linear elution was performed with 20 CV, 50% Elution buffer (20 mM Tris-HCl, 1.0 M NaCl, pH 8.0), and the peaks eluted with about 34% elution buffer were collected. The protein purified with ion-exchanged was further purified with a molecular sieve column, eluted with buffer (20 mM NaCl, 20 mM Tris-HCl, pH 7.5) for 120 CV, and the peaks at about 90 min were collected. The collected elution peaks were detected by SDS-PAGE, and the detection result was shown in FIG. 7. Lane 1 was hGH purified by cSAT; lane 2 was hGH purified by ion exchange column; lane 3 was hGH purified by molecular sieve. Through the two-step purification of ion exchange column and molecular sieve, recombinant human growth hormone hGH protein with purity greater than 99% could be obtained.

### Example 9: RP-HPLC Assay of Human Growth Hormone hGH

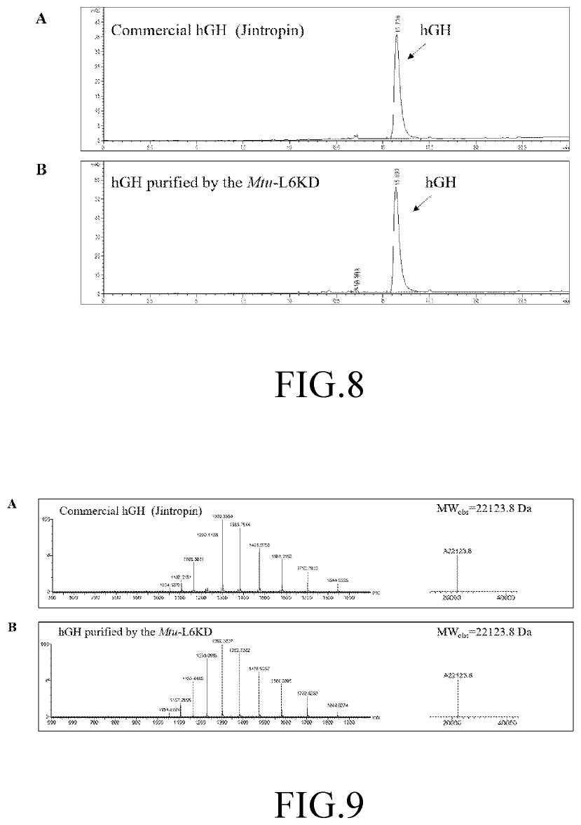

Taking the human growth hormone hGH sample purified by ion exchange column and molecular sieve in Example 8 as an example, RP-HPLC was performed. The standard and purified human growth hormone hGH samples were prepared into 0.1 mg/mL hGH samples with sterile water and analyzed by RP-HPLC. The result was shown in FIG. 8. Instrument: Agilent 1260; Chromatographic column: YMC-Pack ODS-A; Mobile phase: solution A 0.1% (v/v) trifluoroacetic acid in acetonitrile, solution B 0.1% (v/v) 0.1% (v/v) v) aqueous trifluoroacetic acid, the gradient used was shown in Table 8; the injection volume was 99 μL, the flow rate was 1 mL/min, and the temperature was 30° C.

TABLE 8
Parameters for setting the mobile phase gradient

| Time | A solution | B solution |
|---|---|---|
| (min) | ratio (% (v/v)) | ratio (% (v/v)) |
| 0 | 5 | 95 |
| 20 | 95 | 5 |
| 22 | 100 | 0 |

### Example 10: Determination of the Molecular Weight of Human Growth Hormone hGH

Taking the human growth hormone hGH sample obtained from L6KD self-aggregating peptide in Experimental Example 6 as an example, the molecular weight was determined. The human growth hormone hGH sample was dialyzed with mobile phase (solution A: solution B=1:1) to prepare 2 mg/mL hGH sample, and the molecular weight was analyzed by HPLC-MS. Instrument: Agilent 1260 HPLC connected to Waters SYNAPT G2-S time-of-flight mass spectrometry system; Chromatographic column: Acquity UPLC BEH C18 column (2.1 mm×100 mm, 1.7 μm particle size, 130 Å, Waters, USA); Mobile phase: solution A 0.1% (v/v) aqueous formic acid, solution B 0.1% (v/v) formic acid in acetonitrile, the gradient used was shown in Table 9; the injection volume was 10 μL, the flow rate was 0.4 mL/min, and the temperature was 60° C.

TABLE 9
Parameters for setting the mobile phase gradient

| Time | A solution | B solution |
|---|---|---|
| (min) | ratio (% (v/v)) | ratio (% (v/v)) |
| 0 | 75 | 25 |
| 50 | 30 | 70 |
| 55 | 15 | 85 |
| 65 | 15 | 85 |

It can be seen from FIG. 9 that the obtained molecular weight was 22,123.8 Da, which was consistent with the molecular weight of 22,123.8 Da determined with the medical hGH standard (Jintropin), proving that the obtained hGH sequence was correct.

### Example 11: Native-PAGE Assay of Human Growth Hormone hGH

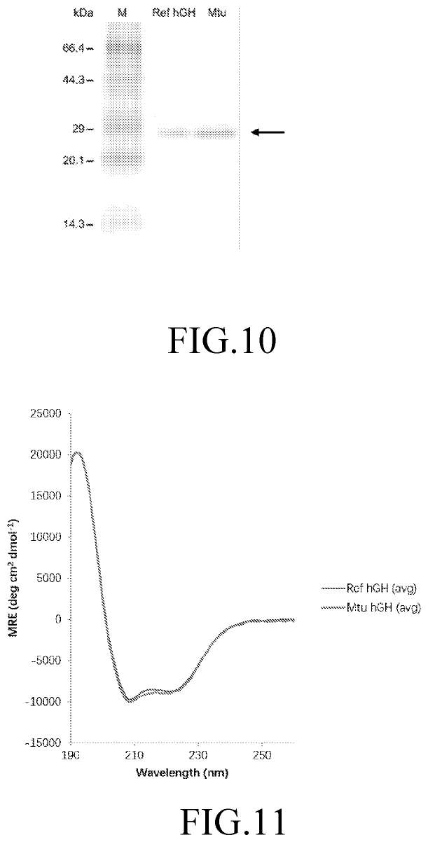

Taking the human growth hormone hGH sample purified by ion exchange column and molecular sieve in Example 8 as an example, the secondary structure was determined. The standard and purified human growth hormone hGH samples were prepared into 0.1 mg/mL hGH samples with sterile water for electrophoresis. The entire electrophoresis process was performed on ice at a voltage of 80 V. The result of Coomassie brilliant blue staining was shown in FIG. 10. It can be seen from FIG. 10 that the structure of hGH purified by cSAT was substantially consistent with that of the medical hGH standard.

### Example 12: Determination of the Secondary Structure of Human Growth Hormone hGH

Taking the human growth hormone hGH sample purified by ion exchange column and molecular sieve in Example 8 as an example, the secondary structure was determined. The standard and purified human growth hormone hGH samples were prepared into 0.1 mg/mL hGH samples with sterile water, and the protein secondary structures of the hGH samples were determined by far ultraviolet circular dichroism analysis. Instrument: Chirascan™ circular dichroism spectrometer. Before the determination of the protein samples, 200 μL of distilled water was added to the sample cell to perform a circular dichroism scan in the far ultraviolet region (190 nm-260 nm) and the obtained chromatographic signal was subtracted as the background signal. Scanning parameters used were shown in Table 10.

TABLE 10
Parameters for settings circular dichroism analysis scan

| | |
|---|---|
| Pathlength | 10 mm |
| Scan speed | 2.5 s/point |
| Temperature | 25° C. |
| Repeat | 3 repeats per sample |

It can be seen from FIG. 11 that the obtained secondary structure analysis chromatogram was substantially consistent with that of the medical hGH standard, proving that the secondary structure of the obtained hGH was correct.

### Example 13: Construction of Human Interferon α2a Fusion Protein Expression Vector

The construction procedure of the expression vectors pET32-L6KD-Mtu ΔI-CM-IFNα2a, pET32-L6KD-Mtu ΔI-CM mutant 1-IFNα2a, pET32-L6KD-Mtu ΔI-CM mutant 2-IFNα2a, pET32-L6KD-Mtu ΔI-CM mutant 3-IFNα2a used in the present Examples were as follows. Taking the construction of pET32-L6KD-Mtu ΔI-CM-IFNα2a as an example, the required primers were designed by oligo 6 and synthesized by Shanghai Sangon Biotechnology Co., Ltd., and were shown in Table 11.

TABLE 11
Oligonucleotide primers used
in this Example

| | | SEQ |
|---|---|---|
| | | ID |
| Primer | Nucleotide sequence | NO |
| J20016-PT-F | 5′-CTGCTGCTGAAAGATCCAACCC | 18 |
| | C-3′ | |
| J20017-Mtu-R | 5-GCAGGTCGCAGTTATGAACCACA | 19 |
| | ACGCCTTCCGCA-3′ | |
| J20018-IFN-F | 5′-TGTGGTTCATAACTGCGACCTG | 20 |
| | CCGCAGAC-3′ | |
| J20019-IFN-R | 5′-TTAGCAGCCGGATCTCAGTGG | 21 |
| | T-3′ | |
| J20020-Term-F | 5′-ACCACTGAGATCCGGCTGCTAA | 22 |
| | CAAAG-3′ | |
| J20003-Or-R | 5′-GCGGTATCAGCTCACTCAAAG | 23 |
| | GCGGTAATACGG-3′ | |
| J20004-Bom-F | 5′-CCTTTGAGTGAGCTGATACCG | 24 |
| | CTCGCCGCAGCCGAAC-3′ | |
| J20015-RBS-R | 5′-GGGTTGGATCTTTCAGCAGCA | 25 |
| | GCAGCAGCAGCATATGT-3′ | |

Firstly, pET32-L6KD-Mtu ΔI-CM-hGH was used as template, J20016-PT-F and J20017-Mtu-R were used as primers, the L6KD-Mtu ΔI-CM polynucleotide fragment was amplified by PCR reaction. The Q5 polymerase from NEB was used in the PCR reaction, and the PCR conditions were: 98° C. 30 sec, 98° C. 10 sec, 72° C. 30 sec, 72° C. 1 min, 30 cycles in total; finally, 72° C. 2 min. After the reaction was completed, the PCR amplification products were separated and recovered by 1% agarose gel. The polynucleotide sequence of human interferon α2a (NCBI No: NM_000605.4) was obtained from NCBI, codon-optimization in E. coli and synthesis were performed by Shanghai Sangon Biotechnology Co., Ltd. The human interferon α2a polynucleotide was obtained by PCR amplification using the synthesized gene as template and J20018-IFN-F and J20019-IFN-R as primers. The Q5 polymerase from NEB (New England Biolab (NEB)) was used in the PCR reaction, and the PCR conditions were: 98° C. 30 sec; 98° C. sec, 72° C. 30 sec, 72° C. 1 min, 30 cycles in total; finally, 72° C. 2 min. After the reaction was completed, the PCR amplification products were separated and recovered by 1% agarose gel.

By adding primers J20016-PT-F and J20019-IFN-R, the two fragments IFNα2a, L6KD-Mtu ΔI-CM were subjected to overlapping PCR reaction: 98° C. 30 sec; 98° C. 10 sec, 72° C. 1 min, 72° C. 2 min, 30 cycles in total; finally, 72° C. 2 min. After the reaction was completed, the PCR amplification products were detected by electrophoresis, and the result showed that the correct bands as expected were amplified by PCR, and then the gel was cut and recovered.

Using pET32-L6KD-Mtu ΔI-CM-hGH as template and J20020-Term-F and J20003-Ori-R as primers, the flori-AmpR-ori polynucleotide fragment was amplified by PCR reaction. Using J20004-Bom-F and J20015-RBS-R as primers, the rop-lacI-T7 promoter-RBS polynucleotide fragment was obtained by PCR amplification. The Q5 polymerase from NEB was used in the PCR reaction, and the PCR conditions were: 98° C. 30 sec, 98° C. 10 sec, 72° C. 1 sec, 72° C. 3 min, 30 cycles in total; finally, 72° C. 4 min. After the reaction was completed, the PCR amplification products were separated and recovered by 1% agarose gel.

The two polynucleotide fragments recovered and amplified as overlapping PCR products were subjected to Gibson assembly at 50° C. for 1 h. The ligated product was transformed into E. coli DH5α competent cells, and the transformed cells were spread on a LB plate supplemented with 100 μg/mL carbenicillin to screen for positive clones. The plasmids were extracted with a plasmid extraction kit and sequenced. The sequencing result showed that the constructed pET32-L6KD-Mtu ΔI-CM-IFNα2a plasmid was correct.

The sequenced and correct plasmids were then transformed into BL21(DE3) (Novagen) competent cells, and the transformed cells were spread on a LB plate supplemented with 100 μg/mL carbenicillin to screen positive clones for subsequent expression and purification. Similar procedures were used to obtain pET32-L6KD-Mtu ΔI-CM mutant 1-IFNα2a, pET32-L6KD-Mtu ΔI-CM mutant 2-IFNα2a, pET32-L6KD-Mtu ΔI-CM mutant 3-IFNα2a plasmids and the expression strains thereof. The structure of the constructed pET32-L6KD-Mtu ΔI-CM-IFNα2a plasmid was shown in FIG. 5B.

### Example 14: Expression and Purification of Human Interferon α2a Fusion Protein in LB Liquid Medium

The strains constructed in Example 13 (containing plasmids pET32-L6KD-Mtu ΔI-CM-IFNα2a, pET32-L6KD-Mtu ΔI-CM mutant 1-IFNα2a, pET32-L6KD-Mtu ΔI-CM mutant 2-IFNα2a, pET32-L6KD-Mtu ΔI-CM mutant 3-IFNα2a) were inoculated into LB liquid medium containing 100 μg/mL carbenicillin and cultured in a shaker at 37° C. to log phase (OD₆₀₀=0.4-0.6), a final concentration of 0.2 mM IPTG was added, and the induction was performed at 18° C. for 24 h. The cells were harvested and the bacterial concentration OD₆₀₀ was measured (the amount of cells with OD₆₀₀ of 1 in 1 mL was referred to as 1 OD below).

The cells were resuspended to 20 OD/mL in lysis buffer B1 (2.4 g Tris, 29.22 g NaCl, 0.37 g Na₂EDTA.2H₂O dissolved in 800 mL of water, adjusted to pH 8.5, and made up to 1 L with water), ultrasonic fragmentation (fragmentation conditions: power 200 W, ultrasonic time 3 sec, interval time 3 sec, ultrasonic times 99) was performed. After centrifugation at 4° C., 15,000 g for 20 min, the supernatants and pellets were collected. The pellets were washed with an equal volume of lysis buffer twice, then fully resuspended with an equal volume of cleavage buffer (PBS supplemented with 40 mM Bis-Tris, pH 6.2, 2 mM EDTA) and placed at 25° C. for 24 h, allowing the intein fully self-cleaved. After centrifugation at 4° C., 15,000 g for 20 min, the pellets were resuspended with an equal volume of lysis buffer. The resulting supernatants and pellets were determined with SDS-PAGE together with the pre-cleaved pellets. The result was shown in FIG. 8A-B. Lanes ES, EP, CP, CS were human growth hormone hGH expression and purification samples, respectively. ES: cell lysate supernatants; EP: cell lysate pellets, where clear aggregates of fusion protein expression can be detected; CP: pellets separated after cleavage; CS: supernatants separated after cleavage, where a clear band for human interferon α2a can be detected. Lanes I-IV was the protein quantification standard containing bovine serum albumin BSA with the successive loading amounts of 2.5 μg, 1.25 μg, 0.625 μg, 0.3125 μg.

According to the protein quantitative standard, the target band was analyzed by densitometric analysis using ImageJ gel quantitative analysis software, and the aggregate yield formed by the fusion protein, the yield of human interferon α2a released into the supernatants after intein-mediated self-cleavage, the cleavage efficiency of Mtu ΔI-CM, the recovery rate of human interferon α2a and the purity in the supernatants could be calculated and were shown in Table 9.

TABLE 12
Expression and purification of human interferon α2a

| | Aggregate | | | | |
|---|---|---|---|---|---|
| | expression ^{a} | | | | |
| | (mg/L | Human interferon | | | Human |
| | culture | α2a yield ^{b} (mg/L | Cleavage | Recovery | interferon α2a |
| Fusion protein | solution) | culture solution) | efficiency ^{c} | rate ^{d} (%) | purity (%) |
| L6KD-Mtu- | 147 | 14 | 96 | 20 | 41 |
| IFNα2a | | | | | |
| L6KD-Mtu(1)- | 118 | 3 | 59 | 5 | 25 |
| IFNα2a | | | | | |
| L6KD-Mtu(2)- | 207 | 24 | 90 | 25 | 67 |
| IFNα2a | | | | | |
| L6KD-Mtu(3)- | 194 | 25 | 95 | 27 | 68 |
| IFNα2a | | | | | |

^{a} yield of protein aggregate,
^{b} yield of human interferon α2a after intein-mediated self-cleavage (amount of protein produced by E. coli cells per liter of LB medium),
^{c} self-cleavage efficiency mediated by intein = 100% × ( the amount of expressed aggregate before cleavage − the amount of aggregate remained after cleavage)/yield of the aggregate before cleavage,
^{d} recovery rate = 100% × IFN α2a real yield/theoretical yield of human interferon α2a produced by protein aggregate with complete cleavage.

The four different fusion proteins as used (L6KD-Mtu-IFNα2a, L6KD-Mtu(1)-IFNα2a, L6KD-Mtu(2)-IFNα2a, L6KD-Mtu(3)-IFNα2a) all existed in the form of precipitate and the aggregate expression was 446-536 mg/L LB culture solution. Four different fusion proteins were subjected to intein Mtu ΔI-CM self-cleavage, IFNα2a was separated from L6KD-Mtu, the cleavage efficiency was 31-72%, the yield of human interferon α2a released into the supernatants after cleavage was 3-25 mg/L LB culture solution, and the IFNα2a purity recovered after cleavage was 25-68%. L6KD-Mtu(3)-IFNα2a fusion protein showed the highest yield and purity of IFNα2a, that is, the yield of human interferon α2a which was obtained by one-step purification through the present purification technology based on self-aggregating peptide and self-cleavage tag was 25 mg/L LB culture solution wet cell weight, and the purity was 68%.

### Example 15: Expression and Purification of Human Interferon IFNα2a Fusion Protein in Fermentation Medium

The strains constructed in Example 12 were inoculated into fermentation medium containing 100 μg/mL carbenicillin and cultured in a shaker at 37° C. to log phase (OD₆₀₀=0.4-0.6). A final concentration of 0.2 mM IPTG was added, induction was performed at 18° C. for 24 h, the cells were harvested, and the bacterial concentration OD₆₀₀ was measured (the amount of cells with OD₆₀₀ of 1 in 1 mL was referred to as 1 OD below). The fermentation medium components used were shown in Table 3.

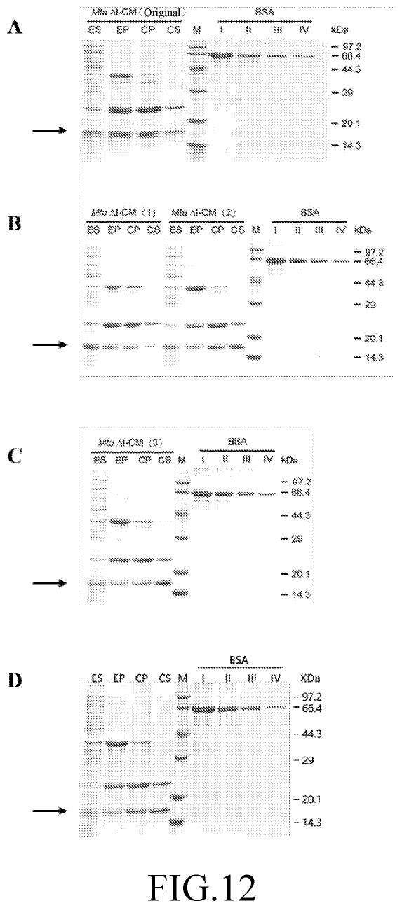

The cells were resuspended to 20 OD/mL in lysis buffer B1 (2.4 g Tris, 29.22 g NaCl, 0.37 g Na₂EDTA.2H₂O dissolved in 800 mL of water, adjusted to pH 8.5, and made up to 1 L with water), and ultrasonic fragmentation (fragmentation conditions: power 200 W, ultrasonic time 3 sec, interval time 3 sec, ultrasonic times 99) was performed. Centrifugation was performed at 4° C., 15,000 g for 20 min, and the supernatants and pellets were collected. The pellets were washed with an equal volume of lysis buffer twice, then fully resuspended with an equal volume of cleavage buffer (PBS supplemented with 40 mM Bis-Tris, pH 6.2, 2 mM EDTA) and placed at 25° C. for 24 h, allowing the intein fully self-cleaved. After centrifugation at 4° C., 15,000 g for 20 min, the pellets were resuspended with an equal volume of lysis buffer. The resulting supernatants and pellets were determined with SDS-PAGE together with the pre-cleaved pellets. The result was shown in FIG. 12D. Lanes ES, EP, CP, CS were human interferon α2a expression and purification samples, respectively. ES: cell lysate supernatants; EP: cell lysate pellets, where clear aggregates of fusion protein expression can be detected; CP: pellets separated after cleavage; CS: supernatants separated after cleavage, where a clear band for human interferon α2a can be detected. Lanes I-IV was the protein quantification standard containing bovine serum albumin BSA with the successive loading amounts were 2.5 μg, 1.25 μg, 0.625 μg, 0.3125 μg.

According to the protein quantitative standard, the target band was analyzed by densitometric analysis using ImageJ gel quantitative analysis software, and the aggregate yield formed by the fusion protein, the yield of human interferon α2a released into the supernatants after intein-mediated self-cleavage, the cleavage efficiency of Mtu ΔI-CM, the recovery rate of human interferon α2a and the purity in the supernatants could be calculated and were shown in Table 13.

TABLE 13
Expression and purification of human interferon α2a in fermentation medium

| | Aggregate | | | | |
|---|---|---|---|---|---|
| | expression ^{a} | | | | |
| | (mg/L | Human interferon | | | Human |
| | culture | α2a yield ^{b} (mg/L | Cleavage | Recovery | interferon α2a |
| Fusion protein | solution) | culture solution) | efficiency ^{c} | rate ^{d} (%) | purity (%) |
| L6KD-Mtu(2)- | 1098 | 90 | 88 | 16 | 50 |
| IFNα2a | | | | | |

^{a} yield of protein aggregate,
^{b} yield of human interferon α2a after intein-mediated self-cleavage (amount of protein produced by E. coli cells per liter of LB medium),
^{c} self-cleavage efficiency mediated by intein = 100% × ( the amount of expressed aggregate before cleavage − the amount of aggregate remained after cleavage)/yield of the aggregate before cleavage,
^{d} recovery rate = 100% × IFN α2a real yield/theoretical yield of human interferon α2a produced by protein aggregate with complete cleavage.

The fusion protein L6KD-Mtu(2)-IFNα2a existed in the form of precipitate and the aggregate expression was 1098 mg/L fermentation culture solution. The fusion protein was subjected to intein Mtu ΔI-CM self-cleavage, IFNα2a was separated from L6KD-Mtu, the cleavage efficiency was 88%, the yield of human interferon α2a released into the supernatants after cleavage was 90 mg/L fermentation culture solution, and the IFNα2a purity recovered after cleavage was 50%. That is, through the present purification technology based on self-aggregating peptide and self-cleavage tag, the yield of human interferon α2a of 90 mg/L fermentation culture solution wet cell weight and the purity of 50% could be obtained in one step.

### REFERENCES

[1] Levarski Z., et al., High-level expression and purification of recombinant human growth hormone produced in soluble form in Escherichia coli. Protein Expression and Purification. 2014. 100, 40-47.
[2] Zamani M., et al., Cloning, expression, and purification of a synthetic human growth hormone in Escherichia coli using response surface methodology. Molecular Biotechnology, 2015. 57, 241-250.
[3] Nguyen M. T., et al., Prokaryotic soluble overexpression and purification of bioactive human growth hormone by fusion to thioredoxin, maltose binding protein, and protein disulfide isomerase. PLoS One. 2014. 9, e89038.
[4] Wang Kuqiang et al., A preparation method of an engineered bacterium expressing high growth hormone and the application thereof, CN201811442625.8
[5] Sen G C, Lengyel P., The interferon system. A bird's eye view of its biochemistry. Journal of Biological Chemistry. 1992. 267, 5017-5020.
[6] Heim M. H., Moradpour D., Blum H. E. C., Expression of Hepatitis C Virus Proteins Inhibits Signal Transduction through the Jak-STAT Pathway. Journal of Virology. 1999. 73, 8469-8475.
[7] Wu W., et al., Active protein aggregates induced by terminally attached self-assembling peptide ELK16 in Escherichia coli. Microbial Cell Factories, 2011. 10, 9
[8] Xing L., et al., Streamlined protein expression and purification using cleavable self-aggregating tags. Microbial Cell Factories, 2011. 10, 42
[9] Zhou B., et al., Small surfactant-like peptides can drive soluble proteins into active aggregates. Microbial Cell Factories, 2012. 11, 10
[10] Lin Zhanglin et al., α helix self-assembling short peptide and the application thereof in protein purification, CN201811557416.8
[11] Zhao Q., et al., Recombinant production of medium-to large-sized peptides in Escherichia coli using a cleavable self-aggregating tag. Microbial Cell Factories, 2016. 15:136
[12] van Pouderoyen G., et al., The crystal structure of Bacillus subtilis lipase: a minimal a/s hydrolase fold enzyme. Journal of Molecular Biology, 2001. 309, 215-226
[13] Collard F., et al., Crystal structure of the deglycating enzyme fructosamine oxidase (Amadoriase II). The Journal of Biological Chemistry, 2008. 283:40
[14] Bray B. L., Large-scale manufacture of peptide therapeutics by chemical synthesis. Nature Reviews Drug Discovery, 2003, 2(7):587-593.
[15] Leader B., et al. Protein therapeutics: a summary and pharmacological classification. Nature Reviews Drug Discovery, 2008, 7(1):21-39
[16] Chen Hao et al., Chinese Journal of Bioengineering, 2002, 22(5):p. 87-92.
[17] Chang C., et al. High-level secretion of human growth hormone by Escherichia coli. Gene, 1987, 55(23): 189-196.
[18] Mooney J. T., et al. Purification of a recombinant human growth hormone by an integrated IMAC procedure. Protein Expression and Purification, 2014: 85-94.
[19] Wood D. W., et al. A genetic system yields self-cleaving inteins for bioseparations. Nature Biotechnology, 1999, 17: 889-892.
[20] Lou S., et al. Peptide Tectonics: Encoded Structural Complementarity Dictates Programmable Self-Assembly. Advanced Science, 2019, 6:1802043

1-40. (canceled)

41. An isolated fusion polypeptide, comprising a target polypeptide moiety and a self-aggregating peptide moiety, wherein the target polypeptide moiety is linked to the self-aggregating peptide moiety via a spacer, wherein the spacer comprises a cleavage site, and wherein the target polypeptide is a polypeptide capable of forming an intramolecular disulfide bond.

42. The fusion polypeptide according to claim 41, wherein the self-aggregating peptide moiety comprises an amphipathic self-assembling short peptide.

43. The fusion polypeptide according to claim 42, wherein the amphipathic self-assembling short peptide is selected from the group consisting of an amphipathic β sheet short peptide, an amphipathic α helix short peptide and a surfactant-like short peptide.

44. The fusion polypeptide according to claim 43, wherein the amphipathic self-assembling short peptide is a surfactant-like short peptide.

45. The fusion polypeptide according to claim 43, wherein the surfactant-like short peptide has 7-30 amino acid residues and has an amino acid sequence as shown in the following formula, from N-terminus to C-terminus:
A-B or B-A
wherein A is a peptide consisting of hydrophilic amino acid residues, the hydrophilic amino acid residues can be identical or different and are selected from the group consisting of Lys, Asp, Arg, Glu, His, Ser, Thr, Asn and Gln;
B is a peptide consisting of hydrophobic amino acid residues, the hydrophobic amino acid residues can be identical or different and are selected from the group consisting of Leu, Gly, Ala, Val, Ile, Phe and Trp;
A and B are linked via a peptide bond; and
wherein the proportion of the hydrophobic amino acid residues in the surfactant-like short peptide is 55%-95%.

46. The fusion polypeptide according to claim 45, wherein the surfactant-like short peptide has 8 amino acid residues, and the proportion of the hydrophobic amino acid residues in the surfactant-like short peptide is 75%.

47. The fusion polypeptide according to claim 43, wherein the surfactant-like short peptide is selected from the group consisting of L6KD, L6KK, L6DD, L6DK, L6K2, L7KD and DKL6.

48. The fusion polypeptide according to claim 43, wherein the surfactant-like short peptide is L6KD, of which the amino acid sequence is shown in SEQ ID NO: 1.

49. The fusion polypeptide according to claim 43, wherein the amphipathic self-assembling short peptide is an amphipathic α helix short peptide.

50. The fusion polypeptide according to claim 49, wherein amphipathic α helix short peptide has a length of 4-30 amino acid residues.

51. The fusion polypeptide according to claim 49, wherein the content of the hydrophobic amino acid residues in the amphipathic α helix short peptide is 40%-80%.

52. The fusion polypeptide according to claim 49, wherein the amphipathic α helix short peptide is α3-peptide, of which the amino acid sequence is shown in SEQ ID NO: 3.

53. The fusion polypeptide according to claim 41, wherein the target polypeptide has a length of 20-400 amino acids, for example, 30-300 amino acids, 35-250 amino acids, 40-200 amino acids.

54. The fusion polypeptide according to claim 41, wherein the target polypeptide moiety is located at C-terminus of the fusion polypeptide.

55. The fusion polypeptide according to claim 41, wherein the target polypeptide is a human growth hormone or Interferon α2a.

56. The fusion polypeptide according to claim 55, wherein the human growth hormone moiety comprises an amino acid sequence as shown in SEQ ID NO:5.

57. The fusion polypeptide according to claim 41, wherein the cleavage site is selected from the group consisting of a temperature dependent cleavage site, a pH dependent cleavage site, an ion dependent cleavage site, an enzyme cleavage site or a self-cleavage site.

58. The fusion polypeptide according to claim 57, wherein the cleavage site is a self-cleavage site.

59. The fusion polypeptide according to claim 41, wherein the spacer is an intein, which comprises a self-cleavage site.

60. The fusion polypeptide according to claim 59, wherein the intein is Mtu ΔI-CM, which comprises a sequence as shown in SEQ ID NO: 27.

61. The fusion polypeptide according to claim 59, wherein the Mtu ΔI-CM is linked to the N-terminus of the target polypeptide moiety.

62. A host cell, comprising the polynucleotide comprising a nucleotide sequence encoding the fusion polypeptide according to claim 41, wherein the host cell is able to express the fusion polypeptide.

63. The host cell according to claim 62, wherein the host cell is a bacterium selected from Escherichia genus, Bacillus genus, Salmonella genus, Pseudomonas genus, and Streptomyces genus.

64. The host cell according to claim 62, wherein the host cell is E. coli.

65. A method for producing and purifying a target polypeptide, comprising the steps of:
(a) culturing the host cell according to claim 62, thereby expressing the fusion polypeptide;
(b) lysing the host cell, removing the soluble fraction of the cell lysate and recovering the insoluble fraction;
(c) releasing the soluble target polypeptide from the insoluble fraction via cleavage of the cleavage site; and
(d) removing the insoluble fraction in step (c) and recovering the soluble fraction containing the target polypeptide.

66. The method according to claim 65, wherein the cleavage is weak acidic pH-mediated self-cleavage.


SEQUENCE LISTING (36 sequences)

SEQ ID NO: 1 - protein, 8 aa, Artificial Sequence
  L6KD
LLLLLLKD

SEQ ID NO: 2 - protein, 8 aa, Artificial Sequence
  EFK8
FEFEFKFK

SEQ ID NO: 3 - protein, 21 aa, Artificial Sequence
  alpha3-peptide
LETLAKALETLAKALETLAKA

SEQ ID NO: 4 - protein, 198 aa, Artificial Sequence
  Mxe GyrA intein
CITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHA

SEQ ID NO: 5 - protein, 191 aa, Artificial Sequence [sequence omitted]

SEQ ID NO: 6 - protein, 15 aa, Artificial Sequence
  GS linker
GGGGSGGGGSGGGGS

SEQ ID NO: 7 - protein, 17 aa, Artificial Sequence
  PT linker
PTPPTTPTPPTTPTPTP

SEQ ID NO: 8 - DNA, 27 bp, Artificial Sequence
  hGH-F primer
cgccatatgttcccgaccatcccgctg

SEQ ID NO: 9 - DNA, 26 bp, Artificial Sequence
  hGH-R primer
gatgcacattcgcatgaaaccgcaag

SEQ ID NO: 10 - DNA, 29 bp, Artificial Sequence
  Mxe-L6KD-F primer
cctaatgtttcatgcgaatgtgcatcacg

SEQ ID NO: 11 - DNA, 50 bp, Artificial Sequence
  Mxe-L6KD-R primer
tgctcgagtcaatctttcagcagcagcagcagcagcggcgtcggggttgg

SEQ ID NO: 12 - DNA, 47 bp, Artificial Sequence
  Mxe-EFK8-R primer
tgctcgagtcacttgaactcgaattcgaactcgaacggcgtcggggt

SEQ ID NO: 13 - DNA, 42 bp, Artificial Sequence
  hGHalpha-R primer
cggactagtgcatctcccgtgatgcacattcgcatgaaaccg

SEQ ID NO: 14 - DNA, 23 bp, Artificial Sequence
  J20001-Mtu-F
ctgctgctgaaagatccaacccc

SEQ ID NO: 15 - DNA, 31 bp, Artificial Sequence
  J19042-Mtu-R
atggtcgggaagttatgaaccacaacgcctt

SEQ ID NO: 16 - DNA, 38 bp, Artificial Sequence
  J19040-hGH-F
ttgtggttcataacttcccgaccatcccgctgtctcgt

SEQ ID NO: 17 - DNA, 22 bp, Artificial Sequence
  J19041-hGH-R
ttagcagccggatctcagtggt

SEQ ID NO: 18 - DNA, 23 bp, Artificial Sequence
  J20016-PT-F
ctgctgctgaaagatccaacccc

SEQ ID NO: 19 - DNA, 35 bp, Artificial Sequence
  J20017-Mtu-R
gcaggtcgcagttatgaaccacaacgccttccgca

SEQ ID NO: 20 - DNA, 30 bp, Artificial Sequence
  J20018-IFN-F
tgtggttcataactgcgacctgccgcagac

SEQ ID NO: 21 - DNA, 22 bp, Artificial Sequence
  J20019-IFN-R
ttagcagccggatctcagtggt

SEQ ID NO: 22 - DNA, 27 bp, Artificial Sequence
  J20020-Term-F
accactgagatccggctgctaacaaag

SEQ ID NO: 23 - DNA, 33 bp, Artificial Sequence
  J20003-Ori-R
gcggtatcagctcactcaaaggcggtaatacgg

SEQ ID NO: 24 - DNA, 37 bp, Artificial Sequence
  J20004-Bom-F
cctttgagtgagctgataccgctcgccgcagccgaac

SEQ ID NO: 25 - DNA, 38 bp, Artificial Sequence
  J20015-RBS-R
gggttggatctttcagcagcagcagcagcagcatatgt

SEQ ID NO: 26 - protein, 165 aa, Artificial Sequence
  hIFN-alpha2a
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 27 - protein, 168 aa, Artificial Sequence
  Mtu deltaI-CM
ALAEGTRIFDPVTGTTHRIEDVVDGRKPIHVVAAAKDGTLHARPVVSWFDQGTRDVIGLRIAGGAILWATPDHKVLTEYGWRAAGELRKGDRVAQPRRFDGFGDSAPIPARVQALADALDDKFLHDMLAEELRYSVIREVLPTRRARTFGLEVEELHTLVAEGVVVHN

SEQ ID NO: 28 - protein, 168 aa, Artificial Sequence
  Mtu deltaI-CM Mut 1
ALAEGTRIFDPVTGTTHRIEDVVDGRKPIHVVAAAKDGTLHARPVVSWFDQGTRDVIGLRIAGGAILWATPDYKVLTEYGWRAAGELRKGDRVAQPRRFDGFGDSAPIPARVQALADALDDKFLHDMLAEELRYSVIREVLPTRRARTFGLEVEELHVLVAEGVVVHN

SEQ ID NO: 29 - protein, 168 aa, Artificial Sequence
  Mtu deltaI-CM Mut 2
ALAEGTRIFDPVTGTTHRIEDVVDGRKPIHVVAAAKDGTLHARPVVSWFDQGTRDVIGLRIAGGAILWATPDVKVLTEYGWRAAGELRKGDRVAQPRRFDGFGDSAPIPARVQALADALDDKFLHDMLAEELRYSVIREVLPTRRARTFGLEVEELHSLVAEGVVVHN

SEQ ID NO: 30 - protein, 168 aa, Artificial Sequence
  Mtu deltaI-CM Mut 3
ALAEGTRIFDPVTGTTHRIEDVVDGRKPIHVVAAAKDGTLHARPVVSWFDQGTRDVIGLRIAGGAILWATPDVKVLTEYGWRAAGELRKGDRVAQPRRFDGFGDSAPIPARVQALADALDDKFLHDMLAEELRYSVIREVLPTRRARTFGLEVEELHCLVAEGVVVHN

SEQ ID NO: 31 - DNA, 495 bp, Artificial Sequence
  hIFN-alpha2a
tgcgacctgccgcagacccactctctgggttctcgtcgtaccctgatgctgctggctcagatgcgtaagatctctctgttctcttgcctgaaagaccgtcacgacttcggtttcccgcaggaagaattcggtaaccagttccagaaagctgaaaccatcccggttctgcacgaaatgatccagcagatcttcaacctgttctctaccaaagactcttctgctgcttgggacgaaaccctgctggacaaattctacaccgaactgtaccagcagctgaacgacctggaagcgtgcgttatccagggtgttggtgttaccgaaaccccgctgatgaaagaagactctatcctggctgttcgtaaatacttccagcgtatcaccctgtacctgaaagagaagaaatactctccgtgcgcttgggaagttgttcgtgctgaaatcatgcgttctttctctctgtctaccaacctgcaggaatctctgcgttctaaagaa

SEQ ID NO: 32 - DNA, 504 bp, Artificial Sequence
  Mtu deltaI-CM
gcgctggctgaaggcacgcgcatttttgatccggtcacgggcacgacgcaccgcattgaagatgttgttgatggccgcaagccgattcatgtggttgcggccgcaaaagatggcaccctgcacgcccgtccggtcgtgagttggtttgatcagggtacgcgtgacgtcattggtctgcgtatcgcgggcggtgcaattctgtgggcaaccccggatcataaagtgctgacggaatatggctggcgtgctgcgggtgaactgcgtaagggtgaccgtgttgcacagccgcgtcgctttgatggcttcggtgacagcgcaccgattccggctcgcgttcaagccctggcagatgctctggatgacaagttcctgcacgacatgctggcggaagaactgcgttactctgttatccgcgaagtcctgccgacccgtcgcgcccgcacgtttggtctggaagtggaagaactgcataccctggttgcggaaggcgttgtggttcataac

SEQ ID NO: 33 - DNA, 504 bp, Artificial Sequence
  Mtu deltaI-CM Mut 1
gcgctggctgaaggcacgcgcatttttgatccggtcacgggcacgacgcaccgcattgaagatgttgttgatggccgcaagccgattcatgtggttgcggccgcaaaagatggcaccctgcacgcccgtccggtcgtgagttggtttgatcagggtacgcgtgacgtcattggtctgcgtatcgcgggcggtgcaattctgtgggcaaccccggattataaagtgctgacggaatatggctggcgtgctgcgggtgaactgcgtaagggtgaccgtgttgcacagccgcgtcgctttgatggcttcggtgacagcgcaccgattccggctcgcgttcaagccctggcagatgctctggatgacaagttcctgcacgacatgctggcggaagaactgcgttactctgttatccgcgaagtcctgccgacccgtcgcgcccgcacgtttggtctggaagtggaagaactgcatgttctggttgcggaaggcgttgtggttcataac

SEQ ID NO: 34 - DNA, 504 bp, Artificial Sequence
  Mtu deltaI-CM Mut 2
gcgctggctgaaggcacgcgcatttttgatccggtcacgggcacgacgcaccgcattgaagatgttgttgatggccgcaagccgattcatgtggttgcggccgcaaaagatggcaccctgcacgcccgtccggtcgtgagttggtttgatcagggtacgcgtgacgtcattggtctgcgtatcgcgggcggtgcaattctgtgggcaaccccggatgttaaagtgctgacggaatatggctggcgtgctgcgggtgaactgcgtaagggtgaccgtgttgcacagccgcgtcgctttgatggcttcggtgacagcgcaccgattccggctcgcgttcaagccctggcagatgctctggatgacaagttcctgcacgacatgctggcggaagaactgcgttactctgttatccgcgaagtcctgccgacccgtcgcgcccgcacgtttggtctggaagtggaagaactgcatagtctggttgcggaaggcgttgtggttcataac

SEQ ID NO: 35 - DNA, 504 bp, Artificial Sequence
  Mtu deltaI-CM Mut 3
gcgctggctgaaggcacgcgcatttttgatccggtcacgggcacgacgcaccgcattgaagatgttgttgatggccgcaagccgattcatgtggttgcggccgcaaaagatggcaccctgcacgcccgtccggtcgtgagttggtttgatcagggtacgcgtgacgtcattggtctgcgtatcgcgggcggtgcaattctgtgggcaaccccggatgttaaagtgctgacggaatatggctggcgtgctgcgggtgaactgcgtaagggtgaccgtgttgcacagccgcgtcgctttgatggcttcggtgacagcgcaccgattccggctcgcgttcaagccctggcagatgctctggatgacaagttcctgcacgacatgctggcggaagaactgcgttactctgttatccgcgaagtcctgccgacccgtcgcgcccgcacgtttggtctggaagtggaagaactgcattgtctggttgcggaaggcgttgtggttcataac

SEQ ID NO: 36 - protein, 41 aa, Artificial Sequence
  TZ1H
EIAQHEKEIQAIEKKIAQHEYKIQAIEEKIAQHKEKIQAIK